(19) United States
(12) Patent Application Publication
(10) Pub. No.: US 2024/0327469 A1
(43) Pub. Date: Oct. 3, 2024

(54) FUNCTIONALISED POLYPEPTIDES, NANOPARTICLES AND USES THEREOF

(71) Applicant: AGENCY FOR SCIENCE, TECHNOLOGY AND RESEARCH, Singapore (SG)

(72) Inventors: Yi Yan YANG, The Nanos (SG); Shengcai YANG, The Nanos (SG); Wei Ping Eddy TAN, The Nanos (SG)

(21) Appl. No.: 18/580,539

(22) PCT Filed: Jul. 22, 2022
(86) PCT No.: PCT/SG2022/050519

§ 371 Date: Jan. 18, 2024

(30) Foreign Application Priority Data
Jul. 23, 2021 (SG) 10202108073W

(51) Int. Cl. C07K 14/01; A61K 9/14; A61K 38/00; A61P 31/00; A61P 35/00

(52) U.S. Cl. CPC C07K 14/01; A61K 9/14; A61P 31/00; A61P 35/00; A61K 38/00

(57) ABSTRACT

The present disclosure relates to functionalised polypeptides, nanoparticles and their 5 uses thereof. The functionalised polypeptide comprises polylysine functionalised with guanidinium moieties, wherein the polypeptide is about 50% to about 98% functionalised. The number of guanidinium functionalised lysine monomeric units is 5 to 100, and the number of lysine monomeric units is 1 to 50. The functionalised polypeptides and nanoparticles can be used for treating a microbial infection or for 10 treating cancer.

### TECHNICAL FIELD

The present invention relates, in general terms, to functionalised polypeptides, nanoparticles and their uses thereof.

### BACKGROUND

Cancer is typically treated with either chemotherapy and/or radiation therapy. While often effective to destroy a significant amount of tumour cells, such therapies often leave behind a number of tumour cells that are resistant to the treatment. These resistant cells can proliferate to form new tumours that are then resistant to treatment.

The use of known combinations of chemotherapeutic drugs has on occasion given rise to multidrug resistant (‘MDR’) tumour cells.

Further, many diseases including cancer are driven by complex molecular interactions that dysregulate several pathways at any one time. Hence therapeutically targeting a single component in one of the pathways may not be sufficient to disrupt or rectify these complex mechanisms. Due to the scale of complexity, early drug discovery studies have increasingly evolved to target multiple molecules, pathways, or networks. In the treatment of such diseases, drugs with different mechanisms may be combined (i.e, combination therapies) with beneficial effects including the effective treatment of MDR tumour cells and the minimisation of side effects such as undesirable cytotoxicity.

However, such drug cocktails are difficult to predict and formulate and are often abandoned in late stage trials.

The widespread usage of chemotherapeutic compounds also induces drug resistance, which further limits the application of chemotherapy. Hence, there is an urgent need to find alternative ways to fight against cancer without inducing drug resistance.

Recently, positive-charged polymers are attracting attentions as macromolecular chemotherapeutic agents as the cancer cell membranes have a net negative charge.

However, cationic polymers exhibit significant instability and toxicity after intravenous administration in vivo. The cationic polymers may electrostatically interact with many negatively charged blood components and lead to the formation of aggregates and vascular occlusion, which greatly hampers their clinical application.

It would be desirable to overcome or ameliorate at least one of the above-described problems.

### SUMMARY

The present invention provides a functionalised polypeptide of Formula (I) or a pharmaceutically acceptable salt, solvate, stereoisomer or prodrug thereof, comprising:
a) monomeric units of Formula (A)

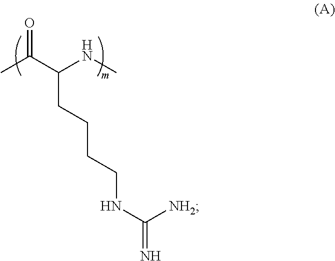

b) at least one monomeric unit of Formula (B)

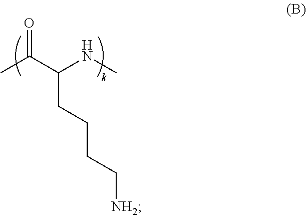

wherein
m is an integer from 5 to 100; and
k is an integer from 1 to 20;
wherein a percentage of monomeric units of Formula (A) relative to the functionalised polypeptide of Formula (I) is about 50% to about 98%.

In some embodiments, the monomeric units of Formula (A) and at least one monomeric unit of Formula (B) form a random co-polypeptide.

In some embodiments, the functionalised polypeptide of Formula (I) is characterised by percentage of monomeric units of Formula (A) relative to the functionalised polypeptide of Formula (I) of about 95%.

In some embodiments, the functionalised polypeptide of Formula (I) further comprises at least one monomeric unit of Formula (C):

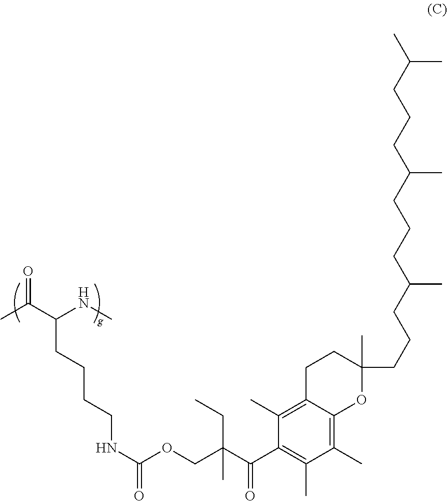

wherein g is an integer from 1 to 10.

In some embodiments, g is 3 or 4.

In some embodiments, the functionalised polypeptide of Formula (I) is characterised by a percentage of monomeric units of Formula (C) relative to the functionalised polypeptide of Formula (I) of about 5% to about 20%, or preferably about 10% to about 16%.

In some embodiments, the monomeric units of Formula (C), monomeric units of Formula (A) and at least one monomeric unit of Formula (B) form a random co-polypeptide.

In some embodiments, m is 18, k is 2, and g is 2. In some embodiments, m is 30, k is 3, and g is 2.

In some embodiments, when monomeric units of Formula (C) are present, the functionalised polypeptide of Formula (I) is characterised by a micelle diameter of about 10 nm to about 200 nm, or preferably about 20 nm to about 50 nm.

In some embodiments, when monomeric units of Formula (C) are present, the functionalised polypeptide of Formula (I) is characterised by a critical micelle concentration (CMC) value of about 1 μg/mL to about 100 μg/mL, or preferably about 8 μg/mL to about 11 μg/mL.

In some embodiments, when monomeric units of Formula (C) are present, the functionalised polypeptide of Formula (I) is characterised by a zeta potential (ζ) of about +1 mV to about +30 mV, or preferably about +8 mV to about +15 mV.

In some embodiments, the functionalised polypeptide of Formula (I) further comprises monomeric units of Formula (D):

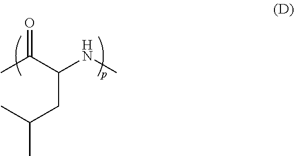

wherein p is an integer from 5 to 100.

In some embodiments, p is 12.

In some embodiments, the monomeric units of Formula (D), the monomeric units of Formula (A) and at least one monomeric unit of Formula (B) form a random co-polypeptide.

In some embodiments, m is 27, k is 2, and p is 12.

In some embodiments, the functionalised polypeptide of Formula (I) is characterised by a degree of polymerisation of 5 to 100, or preferably 20 to 40.

In some embodiments, the functionalised polypeptide of Formula (I) is characterised by a polydispersity index (PDI) of about 1.1 to about 1.7, or preferably about 1.1 to about 1.2.

In some embodiments, the functionalised polypeptide of Formula (I) is characterised by a stability in an aqueous medium of at least 100 days.

In some embodiments, the functionalised polypeptide of Formula (I) is characterised by an antimicrobial activity against Gram-positive bacteria, Gram-negative bacteria and/or fungi.

In some embodiments, the Gram-positive bacteria is selected from S. aureus and MRSA, the Gram-negative bacteria is selected from E. coli, A. baumannii, P. aeruginosa and K. pneumonia, and the fungi is C. albican.

In some embodiments, the functionalised polypeptide of Formula (I) is characterised by an antimicrobial activity with a minimum inhibition concentration (MIC) of about 1 μg/mL to about 200 μg/mL, or preferably about 15 μg/mL to about 130 μg/mL.

In some embodiments, the functionalised polypeptide of Formula (I) is characterised by a minimum bactericidal concentration (MBC) of about 15 μg/mL to about 2000 μg/mL.

In some embodiments, the functionalised polypeptide of Formula (I) is characterised by a minimum fungicidal concentration (MFC) of about 15 μg/mL to about 2000 μg/mL.

In some embodiments, the functionalised polypeptide of Formula (I) is characterised by a (MBC or MFC)/MIC ratio (R) of about 1 to about 500, or preferably 4 or less.

In some embodiments, the functionalised polypeptide of Formula (I) is characterised by a minimum haemolytic concentration at 50% lysis (HC₅₀) of about 100 μg/mL to about 8000 μg/mL.

In some embodiments, the functionalised polypeptide of Formula (I) is characterised by a haemolytic concentration (HC₅₀) of about 4000 μg/mL to about 8000 μg/mL.

In some embodiments, the functionalised polypeptide of Formula (I) is characterised by a IC₅₀ value of about 1 μg/mL to about 200 μg/mL against cancer cells, or preferably about 25 μg/mL against cancer cells.

In some embodiments, the functionalised polypeptide of Formula (I) is characterised by an IC₅₀ ratio of human breast cancer cell line (MCF-7) relative to drug-resistant human breast cancer cell line (MCF-7/ADR) of about 1.

The present invention also provides a functionalised polypeptide nanoparticle, comprising:
a) functionalised polypeptides of Formula (I) or a pharmaceutically acceptable salt, solvate, stereoisomer or prodrug thereof as disclosed herein; and
b) functionalised polypeptides of Formula (II) or a pharmaceutically acceptable salt, solvate, stereoisomer or prodrug thereof, comprising
   i) monomeric units of Formula (E):

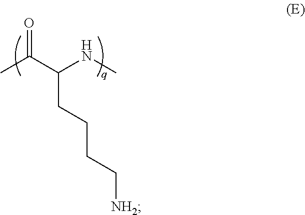

wherein q is an integer from 0 to 10;
   ii) monomeric units of Formula (F):

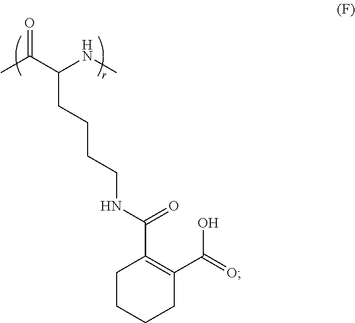

wherein r is an integer from 10 to 100; and
   iii) a methoxy poly(ethylene glycol) (mPEG) terminal end;

wherein a percentage of monomeric units of Formula (F) relative to the functionalised polypeptide of Formula (II) is about 50% to about 98%;

wherein a mole ratio of functionalised polypeptides of Formula (I) to functionalised polypeptides of Formula (II) is about 1:1 to about 1:10.

In some embodiments, q is an integer from 1 to 8.

In some embodiments, r is an integer from 15 to 50.

In some embodiments, q is 6 and r is 20.

In some embodiments, the monomeric units of Formula (E) and monomeric units of Formula (F) form a random co-polypeptide.

In some embodiments, the mPEG has a molecular weight of about 2000 g/mol to about 10000 g/mol.

In some embodiments, the mPEG has about 60 ethylene glycol monomeric units to about 150 ethylene glycol monomeric units, or preferably about 110 ethylene glycol monomeric units.

In some embodiments, the mPEG and the random co-polypeptide comprising monomeric units of Formula (E) and monomeric units of Formula (F) form a block co-polypeptide.

In some embodiments, the functionalised polypeptide of Formula (II) is characterised by percentage of monomeric units of Formula (F) relative to the functionalised polypeptide of Formula (II) of about 80 to about 90%.

In some embodiments, the functionalised polypeptide nanoparticle is dissociable at a pH of about 1 to less than 7.

In some embodiments, the functionalised polypeptide nanoparticle is characterised by an IC₅₀ value of about 1 μg/mL to about 200 μg/mL against cancer cells.

In some embodiments, the functionalised polypeptide nanoparticle is characterised by an IC₅₀ value at a pH of about 6.5 of about 10 μg/mL to about 50 μg/mL against cancer cells or about 25 μg/mL, or preferably at a pH of about 7.4 of about 25 μg/mL to about 150 μg/mL against cancer cells, or about 60 μg/mL.

In some embodiments, the functionalised polypeptide nanoparticle is characterised by an IC₅₀ ratio of human breast cancer cell line (MCF-7) relative to drug-resistant human breast cancer cell line (MCF-7/ADR) of about 1.

The present invention also provide a method of synthesising a functionalised polypeptide of Formula (I), comprising:
a) a monomeric unit of Formula (A)

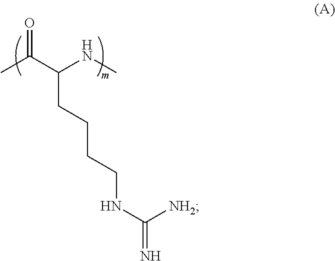

b) a monomeric unit of Formula (B)

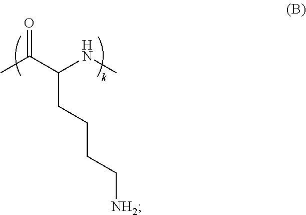

wherein m is an integer from 5 to 100; and k is an integer from 1 to 20;

wherein a percentage of monomeric units of Formula (A) relative to the functionalised polypeptide of Formula (I) is about 50% to about 98%; comprising:
a) reacting a poly(L-lysine) with 2-methyl-2-thiopseudourea in order to form monomeric units of Formula (A).

In some embodiments, the 2-methyl-2-thiopseudourea is protected by a N-protecting group.

In some embodiments, the 2-methyl-2-thiopseudourea is protected by at least one tert-butyloxycarbonyl (Boc) protecting group.

In some embodiments, the 2-methyl-2-thiopseudourea is 1,3-bis(tert-butoxycarbonyl)-2-methyl-2-thiopseudourea.

In some embodiments, when the 2-methyl-2-thiopseudourea is protected by a N-protecting group, the method further comprises a step of deprotecting the guanidinium moiety.

In some embodiments, the method further comprising a step of reacting the poly(L-lysine) with a Vitamin E-functionalized cyclic carbonate of Formula (G) in order to form a monomeric unit of Formula (C):

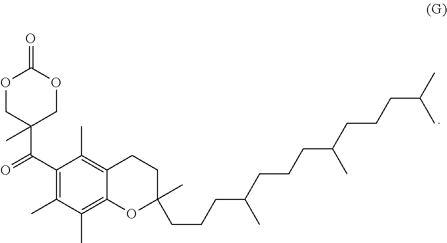

In some embodiments, the method further comprises a step before a) of reacting N-benzyloxycarbonyl-L-lysine-N-carboxyanhydride (Lys(Z)-NCA) monomers under ring opening polymerisation (ROP) conditions and deprotecting the benzyl group in order to form the poly(L-lysine):

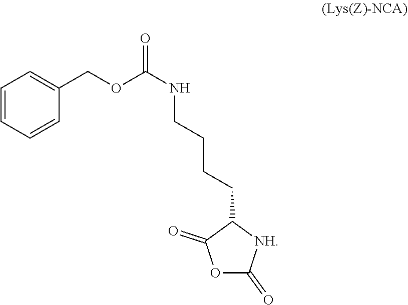

In some embodiments, the step of reacting Lys(Z)-NCA monomers under ROP conditions further comprises L-leucine-N-carboxyanhydride (Leu-NCA) monomers:

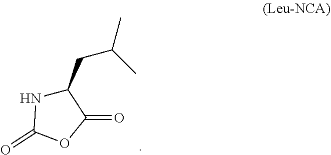

In some embodiments, the ROP is performed with benzylamine as an initiator.

In some embodiments, when Lys(Z)-NCA monomers and Leu-NCA monomers are reacted, the percentage of leucine in the functionalised polypeptide of Formula (I) is about 10% to about 80%, or preferably about 30%.

The present invention also provides a pharmaceutical composition comprising an effective amount of a functionalised polypeptide of formula (I) or a functionalised polypeptide nanoparticle or a pharmaceutically acceptable salt, solvate, stereoisomer or prodrug thereof.

In some embodiments, the pharmaceutical composition further comprises a pharmaceutically acceptable carrier, excipient or diluent.

In some embodiments, the pharmaceutical composition further comprises hydroxypropyl methylcellulose at about 1 wt % to about 5 wt % relative to the pharmaceutical composition.

The present invention also provides a method for treating a microbial infection, the method comprising administering to a subject in need thereof a therapeutically effective amount of a functionalised polypeptide of formula (I) or a functionalised polypeptide nanoparticle or a pharmaceutically acceptable salt, solvate, stereoisomer or prodrug thereof.

In some embodiments, the functionalised polypeptide of formula (I) or a functionalised polypeptide nanoparticle is administrated at a dose of about 0.1 mg/kg to about 300 mg/kg.

In some embodiments, the microbial infection is a bacterial infection or a fungal infection.

The present invention also provides a functionalised polypeptide of formula (I) or a functionalised polypeptide nanoparticle or a pharmaceutically acceptable salt, solvate, stereoisomer or prodrug thereof for use in treating a microbial infection.

The present invention also provides a use of a functionalised polypeptide of formula (I) or a functionalised polypeptide nanoparticle or a pharmaceutically acceptable salt, solvate, stereoisomer or prodrug thereof in the manufacture of a medicament for treating microbial infection.

The present invention also provides a method for treating cancer, the method comprising administering to a subject in need thereof a therapeutically effective amount of a functionalised polypeptide of formula (I) or a functionalised polypeptide nanoparticle or a pharmaceutically acceptable salt, solvate, stereoisomer or prodrug thereof.

In some embodiments, the functionalised polypeptide of formula (I) or a functionalised polypeptide nanoparticle is administrated at a dose of about 0.1 mg/kg to about 300 mg/kg.

In some embodiments, the cancer is breast cancer. In some embodiments, the cancer is a tumorous cancer. In some embodiments, the tumor is suppressed by at least 30%.

The present invention also provides a functionalised polypeptide of formula (I) or a functionalised polypeptide nanoparticle or a pharmaceutically acceptable salt, solvate, stereoisomer or prodrug thereof for use in treating cancer.

The present invention also provides a use of a functionalised polypeptide of formula (I) or a functionalised polypeptide nanoparticle or a pharmaceutically acceptable salt, solvate, stereoisomer or prodrug thereof in the manufacture of a medicament for treating cancer.

### BRIEF DESCRIPTION OF THE DRAWINGS

Embodiments of the present invention will now be described, by way of non-limiting example, with reference to the drawings in which:

FIG. 1 shows a synthesis route of (A) PLL-Gua, and (B) PLL-Gua-VitE.

FIG. 2 shows ¹H NMR spectra of (A) PLL₂₂-Gua(Boc) in CDCl₃ and (B) PLL₂₂-Gua in DMSO-d₆.

FIG. 3 shows ¹H NMR spectra of (A) PLL₂₂-Gua(Boc)-VitE in CDCl₃ and (B) PLL₂₂-Gua-VitE in DMSO-d₆.

FIG. 4 shows a synthesis route of p(lys-r-leu).

FIG. 5 shows a synthesis route of p(lys(Gua)-r-Ieu).

FIG. 6A shows hemolytic activity of PLL, PLL-Gua, PLL-Gua-VitE, p(lys-r-leu) and p(lys(Gua)-r-Ieu).

FIG. 6B shows hemolytic activity of PLL₁₁, PLL₁₁-Gua, and PLL₁₁-Gua-VitE.

FIG. 7A-C shows killing kinetics of PLL₂₂, PLL₂₂-Gua and PLL₂₂-Gua-VitE against S. aureus, E. coli, and C. albicans within 3.0 h.

FIG. 8 shows mechanistic studies of (A) PLL₂₂, (B) PLL₂₂-Gua and (C) PLL₂₂-Gua-VitE at 1×MIC and 2×MIC against β-lactamase SHV-18 producing K. pneumoniae over a period of 4 h. The O.D. value was obtained by subtracting the absorbance value, at 500 nm, at 0 h from the absorbance values, at 500 nm, at each time point. The column bars from left to right at each time point in each bar chart are respectively i) PLL₂₂ or PLL₂₂-Gua or PLL₂₂-Gua-VitE at 1×MIC; ii) PLL₂₂ or PLL₂₂-Gua or PLL₂₂-Gua-VitE at 2×MIC; iii) polymyxin B; and iv) saline.

FIG. 9 shows CLSM images of K. pneumoniae incubated with PLL₂₂, PLL₂₂-Gua and PLL₂₂-Gua-VitE, at 2×MIC, for 1 h.

FIG. 10 shows MBC_{agar} of PLL₂₂-Gua (A) and frequency of resistance to PLL₂₂-Gua (B) against MRSA.

FIG. 11 shows (A) In vivo antibacterial efficacy of PLL₂₂-Gua demonstrated in a MRSA-induced wound infection murine model. (B) H&E staining of skin samples harvested from the control and PLL₂₂-Gua treated mice (200 mg/kg).

FIG. 12 shows images of control mice (right panel) and PLL₂₂-Gua-treated mice (left panel). Treatment with 200 mg/kg of polymer did not affect the normal growth of fur and the body weight of mice from both groups showed no significant difference.

FIG. 13 shows H&E staining of normal tissues from control mice and PLL₂₂-Gua-treated mice. Treatment with polymer did not cause any damage to healthy tissues.

FIG. 14 shows (A) Synthesis routes of mPEG-PLL/CDA. (B) ¹H NMR of mPEG-PLL/CDA in D₂O. (C) Hydrolysis of mPEG-PLL/CDA in D₂O and pH 6.5, D₂O.

FIG. 15A-G shows size change of Gua-NPs in (A) pH 7.4, (B) pH 6.6 and (C) pH 5.4 buffer. (D) The photos of Gua-NPs in de-ionized (DI) water. (E) The zeta potential of Qua-NPs in different pH buffer. (F) The zeta potential of Gua-NPs in different pH buffer. (G) Fluorescence was seen in both AF-488@PLL-Gua solution and AF-488@Gua-NPs solution.

FIG. 16 shows in vitro viability of BT474 cells after 48 h of incubation with (A) mPEG-b-PLL, (B) mPEG-PLL/CDA, (C) PLL₂₂-Gua and (D) Gua-NPs.

FIG. 17 shows in vitro viability of MCF-7 and MCF-7/ADR cells after 48 h of incubation with (A) DOX, (B) PLL₂₂-Gua and (C) Gua-NPs.

FIG. 18 shows in vivo antitumor efficacy of Gua-NPs. (A) Tumor volume as a function of time, (B) final tumor weight and (C) photographs of the dissected tumors by the end of the treatment.

FIG. 19 shows ¹H NMR of PLL₂₂-Gua in D₂O. (A) The PLL₂₂-Gua was incubated in H₂O at room temperature for 6 months. (B) The original PLL₂₂-Gua.

FIG. 20 shows GPC traces of original PLL₂₂-Gua and PLL₂₂-Gua after incubation in H₂O at 37° C. for 100 days.

FIG. 21 shows the viability of HepG-2 human liver carcinoma cells incubated with p(lys-r-leu) or p(lys(Gua)-r-leu) for 48 h.

FIG. 22 shows the killing kinetic curves of p(lys-r-leu) (A) and p(lys(Gua)-r-leu) (B) against BT-474 human breast cancer cell line.

FIG. 23 shows the killing kinetic curves of PLL-Gua and p(lys(Gua)-r-leu) against BT-474 human breast cancer cell line.

FIG. 24 shows the killing kinetic curves of PLL-Gua (A) and Gua-NPs (B) against BT-474 human breast cancer cell line.

FIG. 25 is a scheme showing the preparation and use of the drug-free polypeptide nanoparticles as anticancer agents. The anionic polymer carrier mPEG-b-PLL/CDA and the cationic anticancer polypeptide PLL-Gua self-assembled to form neutrally charged nanoparticles. The nanoparticles were taken up by cells via endocytosis, and released PLLGua from the acidic endosome into the cytosol for PLL-Gua to perform its biological functions as an anticancer agent.

FIG. 26A-I shows viability of BT-474 cells after 48 h-incubation with (A) PLL-Qua, (B) Qua-NPs, (C) PLL-Gua and (D) Gua-NPs. The killing-kinetic curves of (E) PLL-Gua and (F) Gua-NPs at different concentrations. The cell viability was conducted in 6 replicates, and the results were expressed as mean cell viability (%)±SD. PLL-Gua and Gua-NPs had lower IC50 values than PLL-Qua and Qua-NPs, respectively. PLL-Gua and Gua-NPs killed the cancer cells, depending on dose and incubation time. An increased concentration led to faster killing kinetics. Under the same concentration of PLL-Gua and Gua-NPs (50 or 100 μg/mL), PLL-Gua killed the cells more quickly. Apoptotic population of BT-474 cells after 2 h-incubation with PLL-Gua and Gua-NPs at different concentrations (G, I). Annexin V^{Low}PI^{Low}: live cells; Annexin V^{High}pI^{LOW}: early apoptotic cells; Annexin V^{High}pI^{High}: late apoptotic cells; Annexin V^{Low}PI^{High}: necrotic cells. The experiments were conducted in 3 replicates, and the results were expressed as mean apoptosis ratio (%)±SD. ***p<0.001 and ****p<0.0001. PLL-Gua and Gua-NPs induced significant apoptosis rather than necrosis, and a higher population of early apoptotic cells were observed as compared to the control without any treatment. At the higher concentration, a higher population of late apoptotic cells were seen. (H) Confocal microscopic images of BT-474 cells after 30 min-incubation with PLL-Gua (25 μg/mL) and Gua-NPs (25 μg/mL on PLL-Gua basis) (Green: PLL-Gua labelled with AlexaFluor 488; Red: LysoTracker, which stains acidic compartments such as endolysosomes; Blue: Hoechst 33342 stained nucleus). Both PLL-Gua and Gua-NPs entered the cells quickly. Particularly, the cells took up Gua-NPs through endocytosis as evidenced by the yellow regions, which indicates localization of the nanoparticles in the endolysosomes. Scale bar indicates 10 μm.

FIG. 27 shows confocal images of MCF-7 and MCF-7/ADR cells after 24 h treatment with PLL-Gua (25 μg/mL), Gua-NPs (25 μg/mL on PLL-Gua basis) and DOX (5.8 μg/mL). Nuclei stained in blue with Hoechst 33342, ER, nucleoli, and cytoplasmic RNA stained in green with Concanavalin A Alexa Fluor 488 and SYTO 14. Scale bar indicates 20 μm.

FIG. 28 shows viability of MCF-7 and MCF-7/ADR cells after 48 h-incubation with (A) DOX, (B) PLL-Gua or (C) Gua-NPs. The experiments were performed in 6 replicates, and the results were expressed as mean cell viability (%)±SD. MCF-7/ADR cells showed significant resistance towards DOX, while the anticancer efficacy of PLL-Gua and Gua-NPs against MCF-7 and MCF-7/ADR was almost equivalent, suggesting that MCF-7/ADR cells were susceptible to both PLL-Gua and Gua-NPs. (D) Effect of DOX, PLL-Gua and Gua-NPs treatment on migration of MCF-7 cells over 24 h at their respective IC50 concentrations. Both PLL-Gua and Gua-NPs prevented MCF-7 cell migration effectively. Scale bar indicates 100 μm.

FIG. 29 shows photos of mice's tails after intravenous injection with PLL-Gua (10 mg/kg, n=5) and Gua-NPs (20 mg/kg on PLL-Gua basis, n=5) for 6 times on Day 0, 4, 8, 11, 15 and 18. One of five mice in the PLL-Gua treated group died, and one of the mice experienced clotting in the tail vein. All the mice in the Gua-NPs treated group were in good health conditions.

FIG. 30 shows (A-C) blood biochemical analysis on Day 21 of healthy Balb/c mice after intravenous injection with PLL-Gua (10 mg/kg) and Gua-NPs (20 mg/kg on PLL-Gua basis) on Day 0, 4, 8, 11, 15 and 18. (UREA: Blood urea nitrogen; ALT: Alanine Aminotransferase; AST: Aspartate Aminotransferase). (D) LD50 (single lethal dose resulting in 50% mice mortality) values following intravenous injection of PLL-Gua and Gua-NPs. The LD50 of neutrally charged Gua-NPs was 48.9 mg/kg, which was 2.7-fold that of cationic PLL-Gua (17.9 mg/kg). The toxicity of PLL-Gua was decreased after loading with negatively charged carrier mPEG-b-PLL/CDA. (E) Ex vivo fluorescence images of major organs and tumors taken at 6, 24 and 48 h after intravenous injection of Alexa Fluor 750-labelled PLL-Gua (10 mg/kg), and Alexa Fluor 750-labelled Gua-NPs (10 mg/kg on Alexa Fluor 750-labelled PLL-Gua basis). Given its relatively lower in vivo toxicity and higher tumor accumulation, Gua-NPs were chosen for in vivo anticancer efficacy studies.

### DETAILED DESCRIPTION

It was surprisingly found that guanidinium functional polypeptides have an anti-cancer property. Without wanting to be bound by theory, it is believed that such polypeptides can translocated through the cell membrane and interfered with important cellular processes by interacting with intracellular targets, such as proteins and nucleic acids, non-specifically. Since these polypeptides act on multiple intracellular proteins/genes, repeated exposure to them does not induce resistance. The polypeptides are also found to be non-hemolytic. Further, polypeptides are stable in water. This property makes it easy to store and transport the polypeptides, and also to administer the polypeptides. Polypeptides can be prepared via the ring-opening polymerization (ROP) of a-amino acid-N-carboxyanhydride (NCA) monomers. Polypeptides can also be engineered to have unique secondary structures, such as star-shaped, helical and beta-sheet.

Additionally, the functionalised polypeptide was found to have good antimicrobial activity, and in particular good antimicrobial activity against multi-drug resistant (MDR) bacteria. Due to the membrane translocation antimicrobial mechanism, the polypeptide treatment leaves the bacterial membrane intact, avoiding the release of endotoxins.

Bacterial infection is one of the greatest threats to global public health, causing millions of deaths around the world annually. The overuse of antibiotics has led to the onset of multi-drug resistance (MDR) in recent years, further aggravating the problem. To address these issues, researchers have been looking into modifying existing antibiotic classes or introducing new classes with limited cross-resistance, or using adjuvants to sensitize bacteria to antibiotics. In addition, synthetic antimicrobial polymers with different functional mechanisms have been developed. Most antimicrobial polymers kill microbes based on a membrane-disruptive mechanism, which avoids the intrinsic defense mechanism of the microbe and thus mitigates drug resistance development. These polymers are usually amphiphilic in nature, containing both cationic and hydrophobic components. Upon interacting with microbes, these macromolecules are electrostatically attracted to their negatively charged membrane, followed by the insertion of their hydrophobic components. This disrupts and lyses the membrane, eventually leading to cell death. Since they target the microbial membrane, microbes have little chance of developing resistance, even after repeated exposure to these macromolecules. However, membrane disruption might result in the release of endotoxins, which might cause sepsis.

Towards this end, it is believed that guanidinium functional polypeptides can act as antimicrobial agents.

Further, by formulating guanidinium functional polypeptides with negatively charged or anionic polypeptides to form nanoparticles, the positive charges can be further neutralised which reduces the instability and toxicity commonly observed with cationic polymers.

For example, by mixing negatively charged mPEG-PLL/CDA with positively charged polypeptide PLL₂₂-Gua to form pH-sensitive nanoparticles (Gua-NPs) via self-assembly, the positive charge can be further neutralised. These Gua-NPs have excellent stability and exhibit similar anticancer efficacy as compared with PLL₂₂-Gua at low pH. In addition, the PLL₂₂-Gua and Gua-NPs displayed similar activity against MCF-7 and drug-resistant MCF-7/ADR. Moreover, Gua-NPs showed excellent antitumor efficacy in a BT474 human breast cancer xenograft mouse model.

Nanoparticles-based drug delivery systems exhibited a great advantage via the enhanced permeability and retention (EPR) effect of nanoparticles in tumor tissues. Even though the EPR effect improves the accumulation of nanoparticles into tumor sites, poor intracellular release of the loaded cargo may reduce its chemotherapeutic efficacy. Specifically, considering the pH values in different tissues (normal tissues: pH 7.4; tumor extracellular environment: pH 6.2-6.9) and cellular compartments (early endosomes: pH 6.0-7.4; late endosomes: pH 5.5-6.0; lysosomes: pH 4.5-5.5), pH-sensitive nanoparticles such as microenvironment-sensitive nanoparticles can be used to enhance cargo delivery efficacy and improve intracellular drug release. Therefore, in the design of the anionic diblock copolymers, pH-responsive functionality was incorporated in the anionic polypeptide block so that the nanoparticles may release the anticancer polypeptide in tumor tissues and/or in low pH compartments of the cancer cell such as endosomes, and the released cationic polypeptides may bind to the intracellular proteins and genes, halting their bioactivity and in turn, leading to cancer cell death.

The present invention provides a functionalised polypeptide of Formula (I) or a pharmaceutically acceptable salt, solvate, stereoisomer or prodrug thereof, comprising:
a) monomeric units of Formula (A)

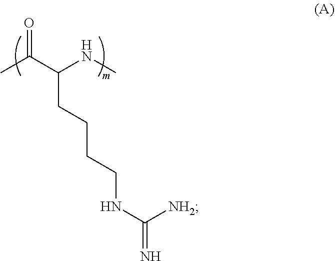

b) at least one monomeric unit of Formula (B)

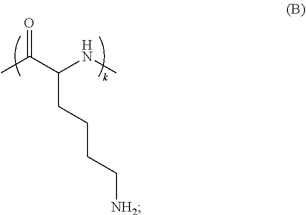

wherein
m is an integer from 5 to 100; and
k is an integer from 1 to 20;
wherein a percentage of monomeric units of Formula (A) relative to the functionalised polypeptide of Formula (I) is about 50% to about 98%.

In some embodiments, the functionalised polypeptide of Formula (I) or a pharmaceutically acceptable salt, solvate, stereoisomer or prodrug thereof comprises:
a) monomeric units of Formula (A)

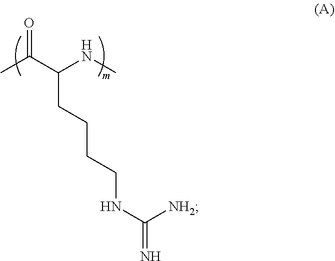

b) at least one monomeric unit of Formula (B)

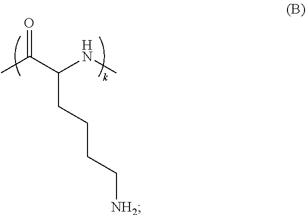

wherein
m is an integer from 5 to 70; and
k is an integer from 1 to 20;
wherein a percentage of monomeric units of Formula (A) relative to the functionalised polypeptide of Formula (I) is about 50% to about 98%.

In some embodiments, the functionalised polypeptide of Formula (I) or a pharmaceutically acceptable salt, solvate, stereoisomer or prodrug thereof, comprises:
a) monomeric units of Formula (A)

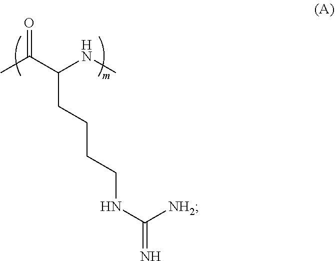

b) at least one monomeric unit of Formula (B)

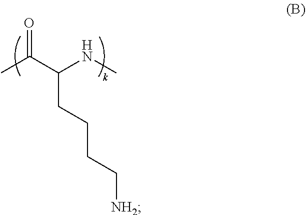

wherein
m is an integer from 8 to 50; and
k is an integer from 1 to 10;
wherein a percentage of monomeric units of Formula (A) relative to the functionalised polypeptide of Formula (I) is about 50% to about 98%.

The presence of the primary amine group in lysine allows further functionalization with other moieties.

A monomer is a molecule that can react together with other monomer molecules to form a larger polymer chain or three-dimensional network in a process called polymerization.

In this sense, monomers are used to form the polymer. The polymer thus comprises of monomeric units linked by covalent bonds.

In some embodiments, m is an integer from 5 to 70, 5 to 65, 5 to 70, 5 to 60, 5 to 55, 5 to 50, 8 to 50, 8 to 45, 8 to 40, 8 to 35, 8 to 30, 9 to 30, 10 to 30, 11 to 30, 12 to 30, 13 to 30, 14 to 30, 15 to 30, 16 to 30, 17 to 30, or 18 to 30.

In some embodiments, k is an integer from 1 to 50, 1 to 45, 1 to 40, 1 to 35, 1 to 30, 1 to 25, 1 to 20, 1 to 19, 1 to 20, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2.

In some embodiments, when m is an integer from 10 to 30, k is an integer from 1 to 10, or 1 to 9, 1 to 8, 1 to 7, 1 to 6, 1 to 5, 1 to 4, 1 to 3, or 1 to 2.

In some embodiments, when m is an integer from 15 to 30, k is an integer from 1 to 10, or 1 to 9, 1 to 8, 1 to 7, 1 to 6, 1 to 5, 1 to 4, 1 to 3, or 1 to 2.

In some embodiments, m is 21 and k is 1.

In some embodiments, m is 33 and k is 2. In some embodiments, m is 33 and k is 1.

In some embodiments, the monomeric units of Formula (A) and at least one monomeric unit of Formula (B) form a random co-polypeptide. In random co-polypeptide or statistical co-polypeptide, the sequence of monomeric units follows a statistical rule, in which the probability of finding a given monomeric unit at a particular point in the chain is equal to the mole fraction of that monomeric unit.

In some embodiments, the functionalised polypeptide of Formula (I) is characterised by percentage of monomeric units of Formula (A) relative to the functionalised polypeptide of Formula (I) of about 50% to about 98%. In other embodiments, the percentage is about 55% to about 98%, about 60% to about 98%, about 65% to about 98%, about 70% to about 98%, about 75% to about 98%, about 80% to about 98%, about 85% to about 98%, or about 95% to about 98%.

In some embodiments, the functionalised polypeptide of Formula (I) is characterised by percentage of monomeric units of Formula (A) relative to the functionalised polypeptide of Formula (I) of about 95%.

In some embodiments, the functionalised polypeptide of Formula (I) further comprises at least one monomeric unit of Formula (C):

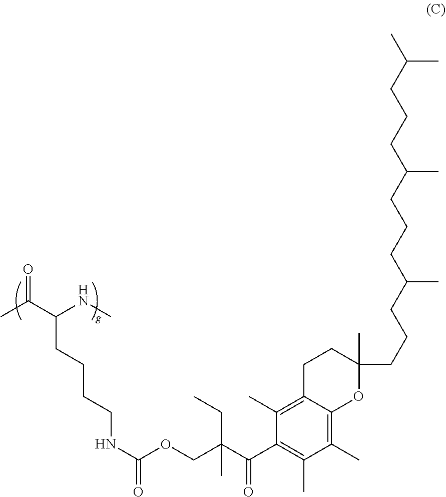

wherein g is an integer from 1 to 10.

In some embodiments, g is an integer from 1 to 10, 1 to 9, 1 to 8, 1 to 7, 1 to 6, 1 to 5, or 1 to 4, or 1 to 2. In some embodiments, g is 3 or 4. In other embodiments, g is 2.

In some embodiments, the functionalised polypeptide of Formula (I) is characterised by a percentage of monomeric units of Formula (C) relative to the functionalised polypeptide of Formula (I) of about 5% to about 20%. In other embodiments, the percentage is about 6% to about 20%, about 7% to about 20%, about 8% to about 20%, about 9% to about 20%, or about 10% to about 20%.

In some embodiments, the functionalised polypeptide of Formula (I) is characterised by a percentage of monomeric units of Formula (C) relative to the functionalised polypeptide of Formula (I) of about 10% to about 16%.

In some embodiments, the monomeric units of Formula (D) is randomly conjugated in the functionalised polypeptide of Formula (I). In some embodiments, the monomeric units of Formula (C), monomeric units of Formula (A) and at least one monomeric unit of Formula (B) form a random co-polypeptide.

In some embodiments, m is 18, k is 1, and g is 3. In some embodiments, m is 18, k is 2, and g is 2.

In some embodiments, m is 30, k is 2, and g is 3. In some embodiments, m is 30, k is 3, and g is 2.

In some embodiments, when monomeric units of Formula (C) are present, the functionalised polypeptide of Formula (I) is characterised by a micelle diameter of about 10 nm to about 200 nm. In other embodiments, the micelle diameter is about 10 nm to about 190 nm, about 10 nm to about 180 nm, about 10 nm to about 170 nm, about 10 nm to about 160 nm, about 10 nm to about 150 nm, about 10 nm to about 140 nm, about 10 nm to about 130 nm, about 10 nm to about 120 nm, about 10 nm to about 110 nm, about 10 nm to about 100 nm, about 10 nm to about 90 nm, about 10 nm to about 80 nm, about 10 nm to about 70 nm, about 10 nm to about 60 nm, about 10 nm to about 50 nm, about 10 nm to about 40 nm, about 10 nm to about 30 nm.

In some embodiments, when monomeric units of Formula (C) are present, the functionalised polypeptide of Formula (I) is characterised by a micelle diameter of about 20 nm to about 200 nm, about 20 nm to about 100 nm, or about 20 nm to about 50 nm.

In some embodiments, when monomeric units of Formula (C) are present, the functionalised polypeptide of Formula (I) is characterised by a critical micelle concentration (CMC) value of about 1 μg/mL to about 100 μg/mL. In other embodiments, the CMC value is about 1 μg/mL to about 90 μg/mL, about 1 μg/mL to about 80 μg/mL, about 1 μg/mL to about 70 μg/mL, about 1 μg/mL to about 60 μg/mL, about 1 μg/mL to about 55 μg/mL, about 1 μg/mL to about 50 μg/mL, about 1 μg/mL to about 45 μg/mL, about 1 μg/mL to about 40 μg/mL, about 1 μg/mL to about 35 μg/mL, about 1 μg/mL to about 30 μg/mL, about 1 μg/mL to about 25 μg/mL, about 1 μg/mL to about 20 μg/mL, about 5 μg/mL to about 20 μg/mL, about 5 μg/mL to about 18 μg/mL, about 5 μg/mL to about 16 μg/mL, about 5 μg/mL to about 15 μg/mL, about 5 μg/mL to about 14 μg/mL, about 5 μg/mL to about 13 μg/mL, or about 5 μg/mL to about 12 μg/mL.

In some embodiments, when monomeric units of Formula (C) are present, the functionalised polypeptide of Formula (I) is characterised by a critical micelle concentration (CMC) value of about 8 μg/mL to about 11 μg/mL.

In some embodiments, when monomeric units of Formula (C) are present, the functionalised polypeptide of Formula (I) is characterised by a zeta potential (ζ) of about +1 mV to about +30 mV. In other embodiments, the zeta potential (ζ) is about +1 mV to about +25 mV, about +1 mV to about +20 mV, about +5 mV to about +20 mV, about +5 mV to about +18 mV, +5 mV to about +16 mV, +5 mV to about +15 mV, +5 mV to about +14 mV, +5 mV to about +13 mV, or +5 mV to about +12 mV.

In some embodiments, when monomeric units of Formula (C) are present, the functionalised polypeptide of Formula (I) is characterised by a zeta potential (ζ) of about +8 mV to about +15 mV.

In some embodiments, the functionalised polypeptide of Formula (I) further comprises monomeric units of Formula (D):

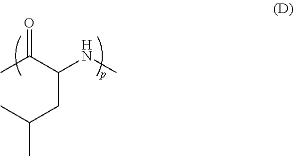

wherein p is an integer from 5 to 100.

Surprisingly, the presence of monomeric units of Formula (D) in the functionalised polypeptide provides for a lower MBCs against E. coli and A. baumannii than those without, demonstrating the presence of hydrophobic leucine leads to stronger antimicrobial activity. The incorporation of hydrophobic leucine amino acid in the functionalised polypeptide can also lead to more rapid cancer cell killing.

In some embodiments, p is an integer from 5 to 100, 5 to 50, 5 to 40, 5 to 30, 5 to 29, 5 to 28, 5 to 27, 5 to 26, 5 to 25, 5 to 24, 5 to 30, 5 to 23, 5 to 22, 5 to 21, 5 to 20, 5 to 19, 5 to 18, 5 to 17, 5 to 16, 5 to 15, 5 to 14, 5 to 13, 5 to 12, 6 to 12, 7 to 12, 8 to 12, 9 to 12, or 10 to 12. In some embodiments, p is 12.

In some embodiments, the monomeric units of Formula (D) is randomly conjugated in the functionalised polypeptide of Formula (I). In some embodiments, the monomeric units of Formula (D), the monomeric units of Formula (A) and at least one monomeric unit of Formula (B) form a random co-polypeptide.

In some embodiments, m is 27, k is 2, and p is 12.

In some embodiments, the functionalised polypeptide of Formula (I) is characterised by a degree of polymerisation of 5 to 100. The degree of polymerization (DP) is defined as the number of monomer units in the polymer. In other embodiments, the degree of polymerisation is 5 to 60, 5 to 55, 5 to 50, 5 to 45, 5 to 40, 5 to 35, 5 to 30, 5 to 25, 5 to 20, 9 to 55, 9 to 50, 9 to 45, 9 to 40, 9 to 35, 10 to 35, 12 to 35, 14 to 35, 16 to 35, 18 to 35, or 20 to 35. In some embodiments, the functionalised polypeptide of Formula (I) is characterised by a degree of polymerisation of 20 to 40, or 22 to 35.

In some embodiments, the functionalised polypeptide of Formula (I) is characterised by a polydispersity index (PDI) of about 1.1 to about 1.7. In some embodiments, the functionalised polypeptide of Formula (I) is characterised by a polydispersity index (PDI) of about 1.1 to about 1.6, about 1.1 to about 1.5, about 1.1 to about 1.4, about 1.1 to about 1.3, or about 1.1 to about 1.2.

In some embodiments, the functionalised polypeptide of Formula (I) is characterised by a stability in an aqueous medium of at least 50 days, 60 days, 70 days, 80 days, 90 days, or 100 days.

In some embodiments, the functionalised polypeptide of Formula (I) is characterised by an antimicrobial activity against Gram-positive bacteria, Gram-negative bacteria and/or fungi/yeast.

In some embodiments, the Gram-positive bacteria is selected from S. aureus and MRSA, the Gram-negative bacteria is selected from E. coli, A. baumannii, P. aeruginosa and K. pneumonia, and the yeast is C. albican.

In some embodiments, the functionalised polypeptide of Formula (I) is characterised by an antimicrobial activity with a minimum inhibition concentration (MIC) value of about 1 μg/mL to about 200 μg/mL. In other embodiments, the MIC is about 10 μg/mL to about 200 μg/mL, about 10 μg/mL to about 190 μg/mL, about 10 μg/mL to about 180 μg/mL, about 10 μg/mL to about 170 μg/mL, about 10 μg/mL to about 160 μg/mL, about 10 μg/mL to about 150 μg/mL, about 10 μg/mL to about 140 μg/mL, about 10 μg/mL to about 130 μg/mL, about 10 μg/mL to about 120 μg/mL, about 10 μg/mL to about 110 μg/mL, or about 10 μg/mL to about 100 μg/mL.

In some embodiments, the functionalised polypeptide of Formula (I) is characterised by an antimicrobial activity with a minimum inhibition concentrations (MICs) of about 15 μg/mL to about 130 μg/mL.

In some embodiments, the functionalised polypeptide of Formula (I) is characterised by a minimum bactericidal concentration (MBC) of about 15 μg/mL to about 2000 μg/mL.

In other embodiments, the MBC is about 15 μg/mL to about 1800 μg/mL, about 15 μg/mL to about 1600 μg/mL, about 15 μg/mL to about 1400 μg/mL, about 15 μg/mL to about 1200 μg/mL, about 15 μg/mL to about 1000 μg/mL, about 15 μg/mL to about 800 μg/mL, about 15 μg/mL to about 600 μg/mL, about 15 μg/mL to about 400 μg/mL, about 15 μg/mL to about 200 μg/mL, or about 15 μg/mL to about 100 μg/mL.

In some embodiments, the functionalised polypeptide of Formula (I) is characterised by a minimum fungicidal concentration (MFC) of about 15 μg/mL to about 2000 μg/mL. In other embodiments, the MFC is about 15 μg/mL to about 1800 μg/mL, about 15 μg/mL to about 1600 μg/mL, about 15 μg/mL to about 1400 μg/mL, about 15 μg/mL to about 1200 μg/mL, about 15 μg/mL to about 1000 μg/mL, about 15 μg/mL to about 800 μg/mL, about 15 μg/mL to about 600 μg/mL, about 15 μg/mL to about 400 μg/mL, about 15 μg/mL to about 200 μg/mL, or about 15 μg/mL to about 100 μg/mL.

In some embodiments, the functionalised polypeptide of Formula (I) is characterised by a (MBC or MFC)/MIC ratio (R) of about 1 to about 500. In other embodiments, the ratio is about 1 to about 400, about 1 to about 300, about 1 to about 200, about 1 to about 150, about 1 to about 100, about 1 to about 80, about 1 to about 70, about 1 to about 65, about 1 to about 60, about 1 to about 55, about 1 to about 50, about 1 to about 45, about 1 to about 40, about 1 to about 35, about 1 to about 30, about 1 to about 25, about 1 to about 20, about 1 to about 15, about 1 to about 10, about 1 to about 5, or about 1 to less than 4.

In some embodiments, the functionalised polypeptide of Formula (I) is characterised by a (MBC or MFC)/MIC ratio (R) of 4 or less.

In some embodiments, the functionalised polypeptide of Formula (I) is characterised by a minimum haemolytic concentration at 50% lysis (HC₅₀) of about 100 μg/mL to about 8000 μg/mL. In other embodiments, the HC₅₀ is about 100 μg/mL to about 6000 μg/mL, about 200 μg/mL to about 6000 μg/mL, about 400 μg/mL to about 6000 μg/mL, about 600 μg/mL to about 6000 μg/mL, about 800 μg/mL to about 6000 μg/mL, about 1000 μg/mL to about 6000 μg/mL, about 1200 μg/mL to about 6000 μg/mL, about 1400 μg/mL to about 6000 μg/mL, about 1600 μg/mL to about 6000 μg/mL, about 1800 μg/mL to about 6000 μg/mL, about 2000 μg/mL to about 6000 μg/mL, about 2500 μg/mL to about 6000 μg/mL, about 3000 μg/mL to about 6000 μg/mL, or about 3500 μg/mL to about 6000 μg/mL.

In some embodiments, the functionalised polypeptide of Formula (I) is characterised by a haemolytic concentration (HC₅₀) of about 4000 μg/mL to about 8000 μg/mL, or about 4000 μg/mL to about 6000 μg/mL.

In some embodiments, the functionalised polypeptide of Formula (I) is characterised by a minimum bactericidal concentration (MBC) against multidrug-resistant bacteria of about 1000 μg/mL to about 2000 μg/mL. In other embodiments, the MBC against multidrug-resistant bacteria is about 1000 μg/mL to about 1900 μg/mL, about 1000 μg/mL to about 1800 μg/mL, about 1000 μg/mL to about 1700 μg/mL, about 1000 μg/mL to about 1600 μg/mL, or about 1000 μg/mL to about 1500 μg/mL.

In some embodiments, the functionalised polypeptide of Formula (I) is characterised by a minimum bactericidal concentration (MBC) against multidrug-resistant bacteria of about 1500 μg/mL.

In some embodiments, the functionalised polypeptide of Formula (I) is characterised by a IC₅₀ value of about 1 μg/mL to about 200 μg/mL against cancer cells. The IC₅₀ value can be measured against cancer cell lines. In other embodiments, the IC₅₀ value of about 1 μg/mL to about 150 μg/mL, about 1 μg/mL to about 100 μg/mL, about 1 μg/mL to about 50 μg/mL, about 5 μg/mL to about 50 μg/mL, about 10 μg/mL to about 50 μg/mL, about 15 μg/mL to about 50 μg/mL, about 20 μg/mL to about 50 μg/mL, about 20 μg/mL to about 45 μg/mL, about 20 μg/mL to about 40 μg/mL, about 20 μg/mL to about 35 μg/mL, or about 20 μg/mL to about 30 μg/mL.

In some embodiments, the functionalised polypeptide of Formula (I) is characterised by a IC₅₀ value of about 25 μg/mL.

In some embodiments, the functionalised polypeptide of Formula (I) is characterised by an IC₅₀ ratio of MCF-7 (human breast cancer cell line) relative to drug resistant MCF-7/ADR of about 1.

The present invention also provides a functionalised polypeptide nanoparticle, comprising:
a) functionalised polypeptides of Formula (I) as disclosed herein; and
b) functionalised polypeptides of Formula (II), comprising
   i) monomeric units of Formula (E):

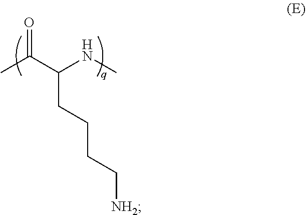

wherein q is an integer from 0 to 10;
   ii) monomeric units of Formula (F):

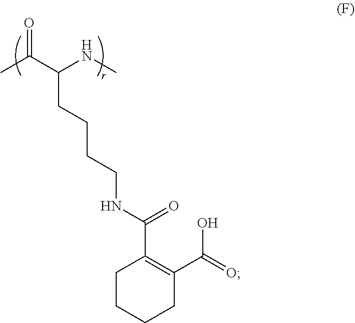

wherein r is an integer from 10 to 100; and
   iii) a methoxy poly(ethylene glycol) (mPEG) terminal end;
wherein a percentage of monomeric units of Formula (F) relative to the functionalised polypeptide of Formula (II) is about 50% to about 98%;
wherein a mole ratio of functionalised polypeptides of Formula (I) to functionalised polypeptides of Formula (II) is about 1:1 to about 1:10.

In some embodiments, the functionalised polypeptide nanoparticle comprises:
a) functionalised polypeptides of Formula (I) as disclosed herein; and
b) functionalised polypeptides of Formula (II), comprising
   i) monomeric units of Formula (E):

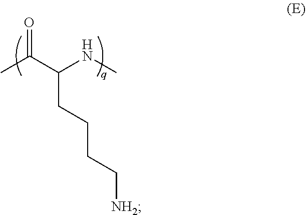

wherein q is an integer from 1 to 10;
   ii) monomeric units of Formula (F):

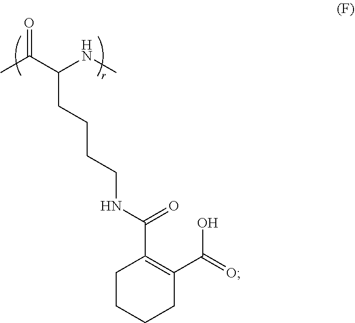

wherein r is an integer from 10 to 80; and
   iii) a methoxy poly(ethylene glycol) (mPEG) terminal end;
wherein a percentage of monomeric units of Formula (F) relative to the functionalised polypeptide of Formula (II) is about 50% to about 98%;
wherein a mole ratio of functionalised polypeptides of Formula (I) to functionalised polypeptides of Formula (II) is about 1:1 to about 1:10.

In some embodiments, the functionalised polypeptide nanoparticle, comprises:
a) functionalised polypeptides of Formula (I) or a pharmaceutically acceptable salt, solvate, stereoisomer or prodrug thereof as disclosed herein; and
b) functionalised polypeptides of Formula (II) or a pharmaceutically acceptable salt, solvate, stereoisomer or prodrug thereof, comprising
   i) monomeric units of Formula (E):

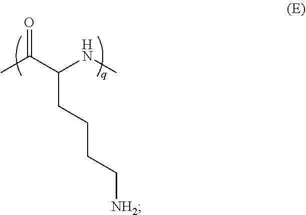

wherein q is an integer from 1 to 10;
   ii) monomeric units of Formula (F):

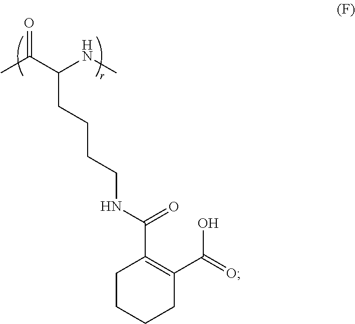

wherein r is an integer from 10 to 30; and
   iii) a methoxy poly(ethylene glycol) (mPEG) terminal end;
wherein a percentage of monomeric units of Formula (F) relative to the functionalised polypeptide of Formula (II) is about 50% to about 90%;
wherein a mole ratio of functionalised polypeptides of Formula (I) to functionalised polypeptides of Formula (II) is about 1:1 to about 1:10.

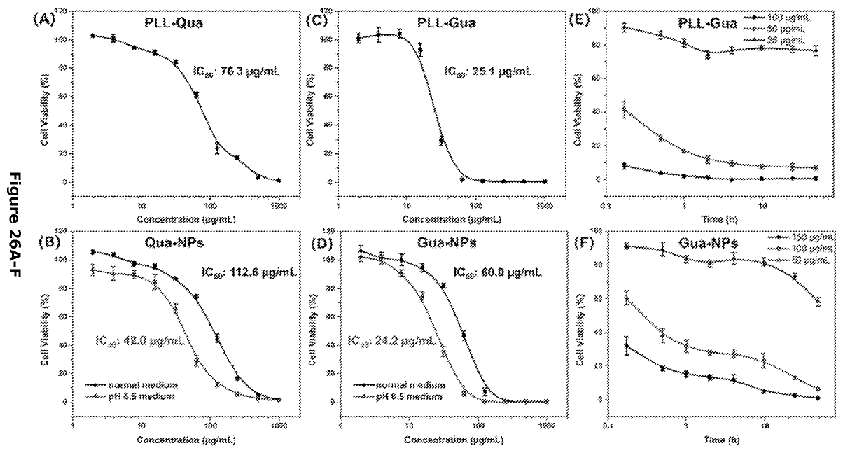

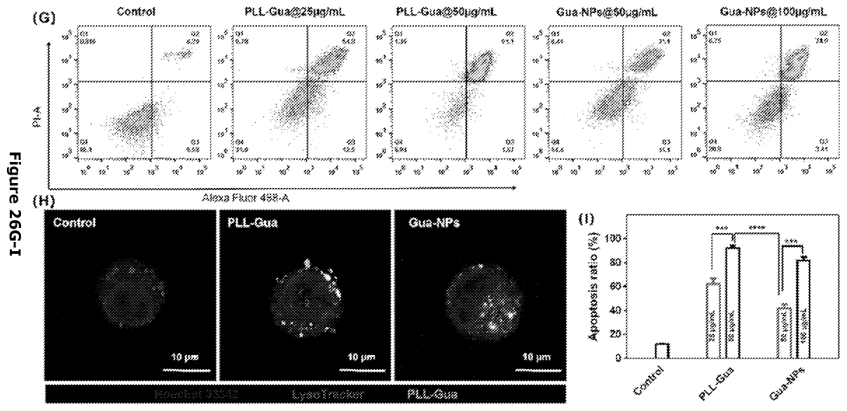

Specifically, anticancer polypeptides with different cationic charges (i.e. primary amine, quaternary ammonium and guanidinium) and diblock copolymer of PEG and pH-sensitive anionic polypeptide were synthesized (FIG. 25). Nanoparticles were formed by simply mixing the cationic and anionic polypeptides via self-assembly, and characterized for particle size, zeta potential, anticancer activity against human cancer cell lines including drug-susceptible and drug-resistant ones, anticancer mechanism (via flow cytometry and confocal microscopy) and hemolytic activity. Their ability to inhibit in vitro migration of cancer cells was also studied. In addition, in vivo toxicity and biodistribution of the nanoparticles were evaluated, and in vivo anticancer efficacy was investigated in a BT-474 human breast cancer xenograft mouse model.

In some embodiments, q is an integer from 1 to 9, 2 to 9, 3 to 9, 4 to 9, or 5 to 9. In some embodiments, q is an integer from 5 to 8.

In some embodiments, r is an integer from 5 to 80, 10 to 75, 5 to 70, 5 to 65, 5 to 60, 5 to 55, 5 to 50, 5 to 45, 5 to 40, 5 to 35, 5 to 30, 10 to 80, 10 to 75, 10 to 70, 10 to 65, 10 to 60, 10 to 55, 10 to 50, 10 to 45, 10 to 40, 10 to 35, 10 to 30, 10 to 29, 10 to 28, 10 to 27, 10 to 26, 11 to 26, 12 to 26, 13 to 26, or 14 to 26. In some embodiments, r is an integer from 15 to 25.

In some embodiments, when q is an integer from 1 to 9, r is an integer from 10 to 50, 10 to 40, 10 to 30, 10 to 29, 10 to 28, 10 to 27, 10 to 26, 11 to 26, 12 to 26, 13 to 26, or 14 to 26. In some embodiments, when q is an integer from 4 to 9, r is an integer from 10 to 50, 10 to 40, 10 to 30, 10 to 29, 10 to 28, 10 to 27, 10 to 26, 11 to 26, 12 to 26, 13 to 26, or 14 to 26. In some embodiments, q is 6 and r is 20.

In some embodiments, the monomeric units of Formula (E) and monomeric units of Formula (F) form a random co-polypeptide.

In some embodiments, the monomeric unit of Formula (F) is a charged monomeric unit.

The charged monomeric unit can be a monomeric unit having an ionised moiety, or having a deprotonated moiety. For example, the monomeric unit of Formula (F) can be:

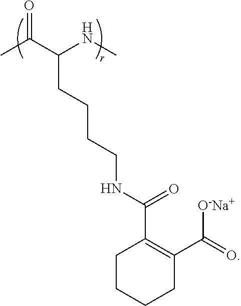

In some embodiments, the mole ratio of ionized or deprotonated moieties to its unionized or protonated counterpart is about 1:1 to about 1:10, about 1:1 to about 1:9, about 1:1 to about 1:8, about 1:1 to about 1:7, about 1:1 to about 1:6, about 1:1 to about 1:5, about 1:1 to about 1:4, about 1:1 to about 1:3, about 1:1 to about 1:2, about 10:1 to about 1:1, about 9:1 to about 1:1, about 8:1 to about 1:1, about 7:1 to about 1:1, about 6:1 to about 1:1, about 5:1 to about 1:1, about 4:1 to about 1:1, about 3:1 to about 1:1, or about 2:1 to about 1:1. In some embodiments, the mole ratio is about 1:1.

In some embodiments, the mole ratio of functionalised polypeptides of Formula (I) to functionalised polypeptides of Formula (II) is about 1:1 to about 1:10, about 1:1 to about 1:9, about 1:1 to about 1:8, about 1:1 to about 1:7, about 1:1 to about 1:6, about 1:1 to about 1:5, about 1:1 to about 1:4, about 1:1 to about 1:3, or about 1:1 to about 1:2. In some embodiments, the mole ratio is about 1:1.

In some embodiments, a mass ratio of functionalised polypeptides of Formula (I) to functionalised polypeptides of Formula (II) is about 10:12 to about 10:30. In other embodiments, the mass ratio is about 10:12 to about 10:28, about 10:12 to about 10:26, about 10:12 to about 10:24, about 10:12 to about 10:22, about 10:12 to about 10:20, about 10:14 to about 10:20, or about 10:16 to about 10:20. In other embodiments, the mass ratio is about 10:18.

In some embodiments, the mPEG has a molecular weight of about 2000 g/mol to about 10,000 g/mol. In other embodiments, the molecular weight is about 3000 g/mol to about 8000 g/mol, about 3000 g/mol to about 7000 g/mol, about 3000 g/mol to about 6000 g/mol, about 3000 g/mol to about 5500 g/mol, about 3000 g/mol to about 5000 g/mol, about 3000 g/mol to about 4500 g/mol, about 3000 g/mol to about 4000 g/mol, or about 3500 g/mol to about 4000 g/mol.

In some embodiments, the mPEG has about 60 ethylene glycol monomeric units to about 150 ethylene glycol monomeric units. In other embodiments, the number of monomeric units is about 65 to about 150, about 70 to about 150, about 75 to about 150, about 80 to about 150, about 85 to about 150, about 90 to about 150, about 95 to about 150, about 100 to about 150, about 100 to about 145, about 100 to about 140, about 100 to about 135, or about 100 to about 130. In some embodiments, the mPEG has about 110 ethylene glycol monomeric units.

In some embodiments, the mPEG and the random co-polypeptide comprising monomeric units of Formula (E) and monomeric units of Formula (F) form a block co-polypeptide. In other embodiments, the mPEG and the random co-polypeptide comprising monomeric units of Formula (E) and monomeric units of Formula (F) form a diblock co-polypeptide. A block co-polypeptide contains two or more different chemical blocks.

In some embodiments, the functionalised polypeptide of Formula (II) is characterised by percentage of monomeric units of Formula (F) relative to the functionalised polypeptide of Formula (II) of about 50% to about 98%. In other embodiments, the percentage is about 55% to about 98%, about 60% to about 98%, about 65% to about 98%, about 70% to about 98%, about 75% to about 98%, about 80% to about 98%, about 85% to about 98%, about 90% to about 98%, about 55% to about 90%, about 60% to about 90%, about 65% to about 90%, about 70% to about 90%, about 75% to about 90%, or about 80% to about 90%.

In some embodiments, the functionalised polypeptide of Formula (II) is characterised by percentage of monomeric units of Formula (F) relative to the functionalised polypeptide of Formula (II) of about 80%.

In some embodiments, the functionalised polypeptide nanoparticle is dissociable at a pH of about 1 to less than 7.

In some embodiments, the functionalised polypeptide nanoparticle is characterised by an IC₅₀ value of about 1 μg/mL to about 200 μg/mL against cancer cells. The IC₅₀ value can be measured against cancer cell lines. In other embodiments, the IC₅₀ value is about 1 μg/mL to about 190 μg/mL, about 1 μg/mL to about 180 μg/mL, about 1 μg/mL to about 170 μg/mL, about 1 μg/mL to about 160 μg/mL, about 1 μg/mL to about 150 μg/mL, about 1 μg/mL to about 140 μg/mL, about 1 μg/mL to about 130 μg/mL, about 1 μg/mL to about 120 μg/mL, about 1 μg/mL to about 110 μg/mL, about 1 μg/mL to about 100 μg/mL, about 1 μg/mL to about 90 μg/mL, about 1 μg/mL to about 80 μg/mL, about 20 μg/mL to about 75 μg/mL, about 20 μg/mL to about 70 μg/mL, about 20 μg/mL to about 65 μg/mL, about 20 μg/mL to about 60 μg/mL, about 20 μg/mL to about 55 μg/mL, about 20 μg/mL to about 50 μg/mL, about 20 μg/mL to about 45 μg/mL, or about 20 μg/mL to about 40 μg/mL.

In some embodiments, the functionalised polypeptide nanoparticle is characterised by an IC₅₀ value at a pH of about 6.5 of about 10 μg/mL to about 50 μg/mL against cancer cells or about 25 μg/mL.

In some embodiments, the functionalised polypeptide nanoparticle is characterised by an IC₅₀ value at a pH of about 7.5 of about 60 μg/mL. In some embodiments, the functionalised polypeptide nanoparticle is characterised by an IC₅₀ value at a pH of about 7.4 of about 25 μg/mL to about 150 μg/mL against cancer cells, or about 60 μg/mL.

In some embodiments, the functionalised polypeptide nanoparticle is characterised by an IC₅₀ ratio of MCF-7 relative to MCF-7/ADR of about 1.

The present invention also provides a method of synthesising a functionalised polypeptide of Formula (I), comprising:
a) a monomeric unit of Formula (A)

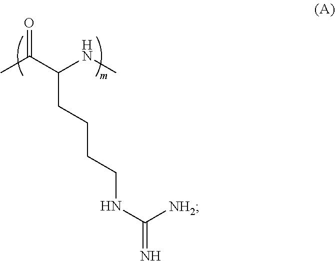

b) a monomeric unit of Formula (B)

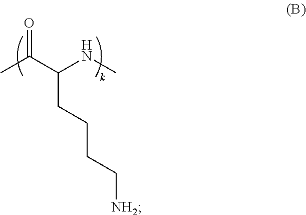

wherein m is an integer from 5 to 100; and k is an integer from 1 to 20;
wherein a percentage of monomeric units of Formula (A) relative to the functionalised polypeptide of Formula (I) is about 50% to about 98%; comprising:
   a) reacting a poly(L-lysine) with 2-methyl-2-thiopseudourea in order to form a monomeric unit of Formula (A).

In some embodiments, the method of synthesising a functionalised polypeptide of Formula (I) comprises:
a) a monomeric unit of Formula (A)

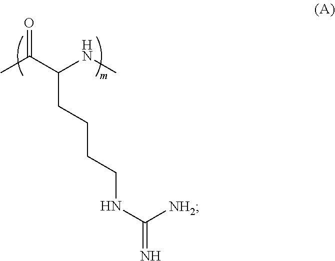

b) a monomeric unit of Formula (B)

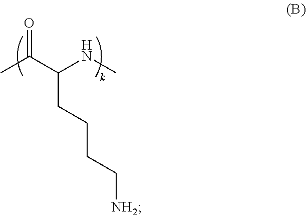

wherein m is an integer from 5 to 70; and k is an integer from 1 to 20;
wherein a percentage of monomeric units of Formula (A) relative to the functionalised polypeptide of Formula (I) is about 50% to about 98%; comprising:
   a) reacting a poly(L-lysine) with 2-methyl-2-thiopseudourea in order to form a monomeric unit of Formula (A).

In some embodiments, a mole ratio of lysine to 2-methyl-2-thiopseudourea is about 1:1 to about 1:10. In other embodiments, the mole ratio is about 1:1 to about 1:9, about 1:1 to about 1:8, about 1:1 to about 1:7, about 1:1 to about 1:6, about 1:1 to about 1:5, or about 1:2 to about 1:5. In other embodiments, the mole ratio is about 1:3.

In some embodiments, the 2-methyl-2-thiopseudourea is protected by a N-protecting group. In some embodiments, the 2-methyl-2-thiopseudourea is protected by at least one tert-butyloxycarbonyl (Boc) protecting group. In some embodiments, the 2-methyl-2-thiopseudourea is 1,3-bis(tert-butoxycarbonyl)-2-methyl-2-thiopseudourea.

In some embodiments, when the 2-methyl-2-thiopseudourea is protected by a N-protecting group, the method further comprises a step of deprotecting the guanidinium moiety.

In some embodiments, the method further comprising a step of reacting the poly(L-lysine) with a Vitamin E-functionalized cyclic carbonate of Formula (G) in order to form a monomeric unit of Formula (C):

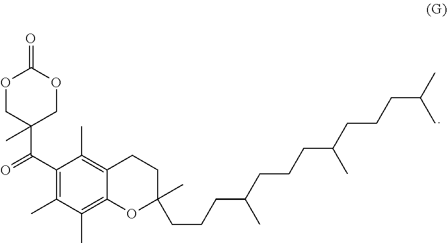

In some embodiments, a mole ratio of poly(L-lysine) to Vitamin E-functionalized cyclic carbonate of Formula (G) is about 1:1 to about 1:10. In other embodiments, the mole ratio is about 1:1 to about 1:9, about 1:1 to about 1:8, about 1:1 to about 1:7, about 1:1 to about 1:6, about 1:1 to about 1:5, or about 1:2 to about 1:5. In other embodiments, the mole ratio is about 1:1.

In some embodiments, the method further comprises a step before a) of reacting N-benzyloxycarbonyl-L-lysine-N-carboxyanhydride (Lys(Z)-NCA) monomers under ring opening polymerisation (ROP) conditions and deprotecting the benzyl group in order to form the poly(L-lysine):

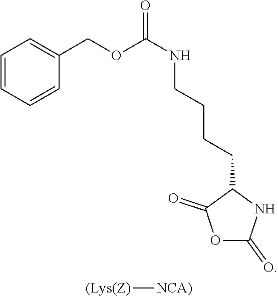

In some embodiments, the step of reacting Lys(Z)-NCA monomers under ROP conditions further comprises L-leucine-N-carboxyanhydride (Leu-NCA) monomers:

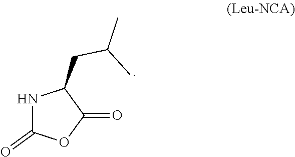

In some embodiments, the ROP is performed with benzylamine as an initiator.

In some embodiments, a mole ratio of Lys(Z)-NCA to Leu-NCA is about 10:1 to about 1:1. In other embodiments, the mole ratio is about 9:1 to about 1:1, about 8:1 to about 1:1, about 7:1 to about 1:1, about 6:1 to about 1:1, about 5:1 to about 1:1, about 4:1 to about 1:1, about 3:1 to about 1:1, or about 2:1 to about 1:1. In other embodiments, the mole ratio is about 2:1.

In some embodiments, when Lys(Z)-NCA monomers and Leu-NCA monomers are reacted, a percentage of leucine in the functionalised polypeptide of Formula (I) is about 10% to about 80%. In other embodiments, the percentage is about 10% to about 70%, about 10% to about 60%, about 10% to about 50%, about 10% to about 45%, about 10% to about 40%, about 10% to about 35%, or about 10% to about 30%.

In some embodiments, when Lys(Z)-NCA monomers and Leu-NCA monomers are reacted, the percentage of leucine in the functionalised polypeptide of Formula (I) is about 30%.

The present invention also provides a pharmaceutical composition comprising an effective amount of a functionalised polypeptide of formula (I) or a functionalised polypeptide nanoparticle or a pharmaceutically acceptable salt, solvate, stereoisomer or prodrug thereof.

In some embodiments, the pharmaceutical composition further comprises a pharmaceutically acceptable carrier, excipient or diluent.

In some embodiments, the pharmaceutical composition further comprises hydroxypropyl methylcellulose at about 1 wt % to about 5 wt % relative to the pharmaceutical composition. In other embodiments, the concentration is about 1 wt % to about 4 wt %, about 1 wt % to about 3 wt %, or about 1 wt % to about 2 wt %.

The present invention also provides a method for treating a microbial infection, the method comprising administering to a subject in need thereof a therapeutically effective amount of a functionalised polypeptide of formula (I) or a pharmaceutically acceptable salt, solvate, stereoisomer or prodrug thereof.

In some embodiments, the functionalised polypeptide of formula (I) is administrated at a dose of about 0.1 mg/kg to about 300 mg/kg. In other embodiments, the dose is 1 mg/kg to about 300 mg/kg, 5 mg/kg to about 300 mg/kg, about 5 mg/kg to about 280 mg/kg, about 5 mg/kg to about 260 mg/kg, about 5 mg/kg to about 240 mg/kg, about 5 mg/kg to about 220 mg/kg, about 5 mg/kg to about 200 mg/kg, about 5 mg/kg to about 180 mg/kg, about 5 mg/kg to about 160 mg/kg, about 5 mg/kg to about 140 mg/kg, about 5 mg/kg to about 120 mg/kg, about 5 mg/kg to about 100 mg/kg, about 5 mg/kg to about 80 mg/kg, or about 5 mg/kg to about 60 mg/kg.

In some embodiments, the microbial infection is a bacterial infection or a fungal infection.

In some embodiments, the microbial infection is a Gram-positive bacteria infection, Gram-negative bacteria infection or a combination thereof.

In some embodiments, the Gram-positive bacteria is selected from S. aureus and MRSA, the Gram-negative bacteria is selected from E. coli, A. baumannii, P. aeruginosa and K. pneumonia, and the fungi is C. albican.

The present invention also provides a functionalised polypeptide of formula (I) or a functionalised polypeptide nanoparticle or a pharmaceutically acceptable salt, solvate, stereoisomer or prodrug thereof for use in treating a microbial infection.

The present invention also provides a use of a functionalised polypeptide of formula (I) or a functionalised polypeptide nanoparticle or a pharmaceutically acceptable salt, solvate, stereoisomer or prodrug thereof in the manufacture of a medicament for treating microbial infection.

The present invention also provides a method for treating cancer, the method comprising administering to a subject in need thereof a therapeutically effective amount of a functionalised polypeptide of formula (I) or a functionalised polypeptide nanoparticle or a pharmaceutically acceptable salt, solvate, stereoisomer or prodrug thereof.

In some embodiments, the functionalised polypeptide of formula (I) is administrated at a dose of about 0.1 mg/kg to about 300 mg/kg. In other embodiments, the dose is 1 mg/kg to about 300 mg/kg, 5 mg/kg to about 300 mg/kg, about 5 mg/kg to about 280 mg/kg, about 5 mg/kg to about 260 mg/kg, about 5 mg/kg to about 240 mg/kg, about 5 mg/kg to about 220 mg/kg, about 5 mg/kg to about 200 mg/kg, about 5 mg/kg to about 180 mg/kg, about 5 mg/kg to about 160 mg/kg, about 5 mg/kg to about 140 mg/kg, about 5 mg/kg to about 120 mg/kg, about 5 mg/kg to about 100 mg/kg, about 5 mg/kg to about 80 mg/kg, or about 5 mg/kg to about 60 mg/kg.

In some embodiments, the cancer is a drug resistant cancer. In some embodiments, the cancer is selected from bladder cancer, breast cancer, colon and rectal cancer, endometrial cancer, kidney cancer, leukemia, liver cancer, lung cancer, melanoma, non-Hodgkin lymphoma, pancreatic cancer, prostate cancer and thyroid cancer. In some embodiments, the cancer is breast cancer.

In some embodiments, the cancer is a tumorous cancer.

In some embodiments, the tumor is suppressed by at least 30%.

The present invention also provides a functionalised polypeptide of formula (I) or a functionalised polypeptide nanoparticle or a pharmaceutically acceptable salt, solvate, stereoisomer or prodrug thereof for use in treating cancer.

The present invention also provides a use of a functionalised polypeptide of formula (I) or a functionalised polypeptide nanoparticle or a pharmaceutically acceptable salt, solvate, stereoisomer or prodrug thereof in the manufacture of a medicament for treating cancer.

In certain embodiments, the pharmaceutically acceptable form is an isomer. The term “isomer” as used herein includes any and all geometric isomers and stereoisomers (e.g., enantiomers, diasteromers, etc.). For example, “isomer” include cis- and trans-isomers, E— and Z-isomers, R— and S-enantiomers, diastereomers, (D)-isomers, (L)-isomers, racemic mixtures thereof, and other mixtures thereof, as falling within the scope of the invention. For instance, an isomer/enantiomer may, in some embodiments, be provided substantially free of the corresponding enantiomer, and may also be referred to as “optically enriched.” “Optically-enriched,” as used herein, means that the compound is made up of a significantly greater proportion of one enantiomer. In certain embodiments the compound of the present invention is made up of at least about 90% by weight of a preferred enantiomer. In other embodiments the compound is made up of at least about 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, N Y, 1962); Wilen, S. H. Tables of Resolving Agents and Optical Resolutions p. 268 (E. L. Eliel, Ed., Univ. of Notre Dame Press, Notre Dame, IN 1972).

It will also be recognised that functionalised polypeptides of the invention may possess asymmetric centres and are therefore capable of existing in more than one stereoisomeric form. The invention thus also relates to functionalised polypeptides in substantially pure isomeric form at one or more asymmetric centres eg., greater than about 90% ee, such as about 95% or 97% ee or greater than 99% ee, as well as mixtures, including racemic mixtures, thereof. Such isomers may be prepared by asymmetric synthesis, for example using chiral intermediates, or mixtures may be resolved by conventional methods, eg., chromatography, or use of a resolving agent.

The functionalised polypeptides of the invention can be administered to a subject as a pharmaceutically acceptable salt thereof. Suitable pharmaceutically acceptable salts include, but are not limited to salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicyclic sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium. In particular, the present invention includes within its scope cationic salts eg sodium or potassium salts, or alkyl esters (eg methyl, ethyl) of the phosphate group.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

It will be appreciated that any functionalised polypeptides that is a prodrug of the functionalised polypeptides of formula (I) is also within the scope and spirit of the invention. Thus the functionalised polypeptides of the invention can be administered to a subject in the form of a pharmaceutically acceptable pro-drug. The term “pro-drug” is used in its broadest sense and encompasses those derivatives that are converted in vivo to the compound of the invention. Such derivatives would readily occur to those skilled in the art. Other texts which generally describe prodrugs (and the preparation thereof) include: Design of Prodrugs, 1985, H. Bundgaard (Elsevier); The Practice of Medicinal Chemistry, 1996, Camille G. Wermuth et al., Chapter 31 (Academic Press); and A Textbook of Drug Design and Development, 1991, Bundgaard et al., Chapter 5, (Harwood Academic Publishers). For example, the amino moiety in lysine can be protected as an ester.

The functionalised polypeptides of the invention may be in crystalline form either as the free compound or as a solvate (e.g. hydrate) and it is intended that both forms are within the scope of the present invention. Methods of solvation are generally known within the art.

The functionalised polypeptides of the invention, or a pharmaceutically acceptable salt, solvate or prodrug thereof is administered to the patient in a therapeutically effective amount. As used herein, a therapeutically effective amount is intended to include at least partially attaining the desired effect, or delaying the onset of, or inhibiting the progression of, or halting or reversing altogether the progression of microbial infection and/or cancer.

As used herein, the term “effective amount” relates to an amount of functionalised polypeptides which, when administered according to a desired dosing regimen, provides the desired therapeutic activity. Dosing may occur at intervals of minutes, hours, days, weeks, months or years or continuously over any one of these periods. Suitable dosages may lie within the range of about 0.1 ng per kg of body weight to 1 g per kg of body weight per dosage, such as is in the range of 1 mg to 1 g per kg of body weight per dosage. In one embodiment, the dosage may be in the range of 1 mg to 500 mg per kg of body weight per dosage. In another embodiment, the dosage may be in the range of 1 mg to 250 mg per kg of body weight per dosage. In yet another embodiment, the dosage may be in the range of 1 mg to 100 mg per kg of body weight per dosage, such as up to 50 mg per body weight per dosage.

Suitable dosage amounts and dosing regimens can be determined by the attending physician and may depend on the severity of the condition as well as the general age, health and weight of the patient to be treated.

The functionalised polypeptides of the invention may be administered in a single dose or a series of doses. While it is possible for the active ingredient to be administered alone, it is preferable to present it as a composition, preferably as a pharmaceutical composition. The formulation of such compositions is well known to those skilled in the art. The composition may contain any suitable carriers, diluents or excipients. These include all conventional solvents, dispersion media, fillers, solid carriers, coatings, antifungal and antibacterial agents, dermal penetration agents, surfactants, isotonic and absorption agents and the like. It will be understood that the compositions of the invention may also include other supplementary physiologically active agents.

The carrier must be pharmaceutically “acceptable” in the sense of being compatible with the other ingredients of the composition and not injurious to the patient. The compositions may conveniently be presented in unit dosage form and may be prepared by any methods well known in the art of pharmacy. Such methods include the step of bringing into association the active ingredient with the carrier which constitutes one or more accessory ingredients. In general, the compositions are prepared by uniformly and intimately bringing into association the active ingredient with liquid carriers or finely divided solid carriers or both, and then if necessary shaping the product.

The functionalised polypeptides may be injected directly to the eye, and in particular the vitreous of the eye. The compound, composition or combination of the invention can be administered to the vitreous of the eye using any intravitreal or transscleral administration technique. For example, the compound, composition or combination can be administered to the vitreous of the eye by intravitreal injection. Intravitreal injection typically involves administering a compound of the invention or a pharmaceutically acceptable salt, solvate or prodrug in a total amount between 0.1 ng to 10 mg per dose.

Injectables for such use can be prepared in conventional forms, either as a liquid solution or suspension or in a solid form suitable for preparation as a solution or suspension in a liquid prior to injection, or as an emulsion. Carriers can include, for example, water, saline (e.g., normal saline (NS), phosphate-buffered saline (PBS), balanced saline solution (BSS)), sodium lactate Ringer's solution, dextrose, glycerol, ethanol, and the like; and if desired, minor amounts of auxiliary substances, such as wetting or emulsifying agents, buffers, and the like can be added. Proper fluidity can be maintained, for example, by using a coating such as lecithin, by maintaining the required particle size in the case of dispersion and by using surfactants. By way of example, the compound, composition or combination can be dissolved in a pharmaceutically effective carrier and be injected into the vitreous of the eye with a fine gauge hollow bore needle (e.g., 30 gauge, ½ or ⅜ inch needle) using a temporal approach (e.g., about 3 to about 4 mm posterior to the limbus for human eye to avoid damaging the lens).

A person skilled in the art will appreciate that other means for injecting and/or administering the functionalised polypeptides, or composition to the vitreous of the eye can also be used. These other means can include, for example, intravitreal medical delivery devices. These devices and methods can include, for example, intravitreal medicine delivery devices, and biodegradable polymer delivery members that are inserted in the eye for long term delivery of medicaments. These devices and methods can further include transscleral delivery devices.

Other modes of administration including topical or intravenous administration may also be possible. For example, solutions or suspensions of the functionalised polypeptides, or composition of the invention may be formulated as eye drops, or as a membranous ocular patch, which is applied directly to the surface of the eye. Topical application typically involves administering the compound of the invention in an amount between 0.1 ng/kg and 1 g/kg.

The functionalised polypeptides, or composition of the invention may also be suitable for intravenous administration. For example, a compound of formula (I) or a pharmaceutically acceptable salt, solvate or prodrug thereof may be administered intravenously at a dose of up to 1 g/kg.

The functionalised polypeptides, or composition of the invention may also be suitable for oral administration and may be presented as discrete units such as capsules, sachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be presented as a bolus, electuary or paste. In another embodiment, the functionalised polypeptides of formula (I) or a pharmaceutically acceptable salt, solvate or prodrug is orally administerable.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g inert diluent, preservative disintegrant (e.g. sodium starch glycolate, cross-linked polyvinyl pyrrolidone, cross-linked sodium carboxymethyl cellulose) surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

The functionalised polypeptides, or composition of the invention may be suitable for topical administration in the mouth including lozenges comprising the active ingredient in a flavoured base, usually sucrose and acacia or tragacanth gum; pastilles comprising the active ingredient in an inert basis such as gelatine and glycerin, or sucrose and acacia gum; and mouthwashes comprising the active ingredient in a suitable liquid carrier.

The functionalised polypeptides, or composition of the invention may be suitable for topical administration to the skin may comprise the compounds dissolved or suspended in any suitable carrier or base and may be in the form of lotions, gel, creams, pastes, ointments and the like. Suitable carriers include mineral oil, propylene glycol, polyoxyethylene, polyoxypropylene, emulsifying wax, sorbitan monostearate, polysorbate 60, cetyl esters wax, cetearyl alcohol, 2-octyldodecanol, benzyl alcohol and water. Transdermal patches may also be used to administer the compounds of the invention.

The functionalised polypeptides, or composition of the invention may be suitable for parenteral administration include aqueous and non-aqueous isotonic sterile injection solutions which may contain anti-oxidants, buffers, bactericides and solutes which render the compound, composition or combination isotonic with the blood of the intended recipient; and aqueous and non-aqueous sterile suspensions which may include suspending agents and thickening agents. The compound, composition or combination may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage composition or combinations are those containing a daily dose or unit, daily sub-dose, as herein above described, or an appropriate fraction thereof, of the active ingredient.

It should be understood that in addition to the active ingredients particularly mentioned above, the composition of this invention may include other agents conventional in the art having regard to the type of composition in question, for example, those suitable for oral administration may include such further agents as binders, sweeteners, thickeners, flavouring agents disintegrating agents, coating agents, preservatives, lubricants and/or time delay agents. Suitable sweeteners include sucrose, lactose, glucose, aspartame or saccharine. Suitable disintegrating agents include cornstarch, methylcellulose, polyvinylpyrrolidone, xanthan gum, bentonite, alginic acid or agar. Suitable flavouring agents include peppermint oil, oil of wintergreen, cherry, orange or raspberry flavouring.

Suitable coating agents include polymers or copolymers of acrylic acid and/or methacrylic acid and/or their esters, waxes, fatty alcohols, zein, shellac or gluten.

Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

### Examples

### Materials

All chemicals were purchased from Sigma-Aldrich and Tokyo Chemical Industry (Singapore) and used as received unless specified. Polymyxin B was purchased from Merck. PLL, with various DP, was synthesized via ROP of Lys(Z)-NCA monomer with n-hexylamine as the initiator and a subsequent deprotection of benzyl groups according to a reported protocol (S. Lv, Z. Tang, D. Zhang, W. Song, M. Li, J. Lin, H. Liu, X. Chen, J. Controlled Release 2014, 194, 220). Vitamin E-functionalized cyclic carbonate monomer (MTC-VitE) was prepared according to our previous protocols(V. W. L. Ng, X. Ke, A. L. Z. Lee, J. L. Hedrick, Y. Y. Yang, Adv. Mater. 2013, 25, 6730). Phosphate-buffered saline (PBS, 10×) was purchased from 1st BASE and diluted to 1×PBS before use. Cation-adjusted Mueller-Hinton broth (MHB) powder was bought from BD Diagnostics and used to prepare the microbial broth according to the manufacturer's instructions. LB Agar Powder, Miller, was bought from Bio Basic and used to prepare LB agar plates according to the manufacturer's instructions. Staphylococcus aureus (ATCC No. 6538), Escherichia coli (ATCC No. 25922), Pseudomonas aeruginosa (ATCC No. 9027), Acinetobacter baumannii (ATCC No. BAA1709), β-lactamase SHV-18 producing Klebsiella pneumoniae subsp. pneumoniae (ATCC No. 700603), Candida albicans (ATCC No. 10231) were purchased from ATCC (U.S.A.) and reconstituted according to the suggested protocols. Methicillin-resistant Staphylococcus aureus RI0309N (MRSA) was isolated from the Infectious Diseases Research Laboratory, the National Centre for Infectious Diseases and Tan Tock Seng Hospital.

### Examples

### Methods

¹H NMR spectra of the polypeptides were recorded on a Bruker AV 400M NMR spectrometer in D₂O, TFA-d, DMSO-d or CDCl₃. Gel permeation chromatography (GPC) analysis of the polypeptides was conducted on a Waters 2414 system equipped with an ultra-hydrogel linear column and a Waters 2414 refractive index detector (eluent: H₂O:CH₃COOH:Methanol: CH₃COONa=1080:460:460:82; flow rate: 0.5 mL/min; temperature: 35° C.; standard: poly(ethylene glycol)). Particle size and zeta potential of the polypeptide samples were measured on a Malvern Zen3690 Zetasizer Nano ZS90 (Malvern Panalytical). Critical micelle concentration (CMC) of the polypeptides was measured by fluorescence spectroscopy, using pyrene as a probe, on a FluoroMax-4 spectrofluorometer (HORIBA Scientific) with an excitation wavelength from 300 nm to 360 nm, and emission wavelength of 395 nm. UV-Vis spectra of the polypeptides were measured on a UV-3600 spectrophotometer (SHIMADZU). The concentration of bacteria was estimated measuring the optical density (OD), at 600 nm, on a Spark 10M multimode microplate reader (TECAN, Switzerland).

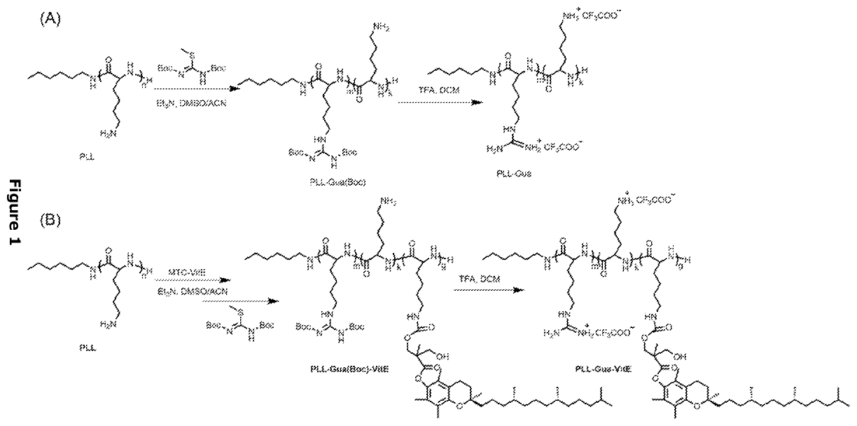

Synthesis of guanidinium-functionalized PLL polymer (PLL-Gua) (FIG. 1a) PLL₂₂ (100 mg) was dissolved in a solution of dimethyl sulfoxide (DMSO, 8.0 mL) and trimethylamine (Et₃N, 1.0 mL). To this solution, 1,3-bis(tert-butoxycarbonyl)-2-methyl-2-thiopseudourea (610 mg) in acetonitrile (ACN, 8.0 mL) was added and stirred at room temperature (RT, ˜22° C.) overnight, under a nitrogen atmosphere. PLL₂₂-Gua(Boc) with DP of 22 was then obtained after dialysis (48 h, MWCO=1000 Da, VMeOH/VDCM, 1/1; MeOH: methanol, DCM: dichloromethane) and removal of solvent in vacuo.

An acid-mediated deprotection strategy was used for the removal of Boc protection group in PLL₂₂-Gua(Boc). In brief, PLL₂₂-Gua(Boc) (100 mg) was dissolved in DCM (9.0 mL) and trifluoroacetic acid (TFA, 1.0 mL), and stirred overnight at RT. The solvent was then removed under vacuum and the crude PLL₂₂-Gua was re-dissolved in MeOH, precipitated in cold diethyl ether for 3 times. Upon the removal of the residual solvent, PLL₂₂-Gua was obtained as a white solid. PLL₃₅-Gua with DP of 35 was synthesized using a similar protocol.

PLL₂₂-Gua(Boc) Yield: 95%; ¹H NMR (400 MHz, CDCl₃, ppm): δ 11.46 (Boc-NH—), 8.26 (—CH₂—CH₂—CH₂—CH₂—NH—), 3.83 (—CH— of backbone), 3.36 (—CH₂—NH— or —CH₂—NH₂ of lysine), 2.09-1.16 (—CH₂—CH₂—CH₂—CH₂—NH— of lysine and —CH₃ of Boc).

PLL₂₂-Gua Yield: 82%; ¹H NMR (400 MHz, DMSO-d, ppm): δ8.25-6.82 (—NH— and —NH₂), 4.23 (—CH— of backbone), 3.04 (—CH₂—NH— or —CH₂—NH₂ of lysine), 1.79-1.12 (—CH₂—CH₂—CH₂—CH₂—NH— of lysine) PLL₃₅-Gua(Boc) Yield: 92%; ¹H NMR (400 MHz, CDCl₃, ppm): δ11.47 (Boc-NH—), 8.27 (—CH₂—CH₂—CH₂—CH₂—NH—), 3.85 (—CH— of backbone), 3.37 (—CH₂—NH— or —CH₂—NH₂ of lysine), 2.12-1.10 (—CH₂—CH₂—CH₂—CH₂—NH— of lysine and —CH₃ of Boc).

PLL₃₅-Gua Yield: 83%; ¹H NMR (400 MHz, DMSO-d, ppm): δ8.25-6.84 (—NH— and —NH₂), 4.24 (—CH— of backbone), 3.06 (—CH₂—NH— or —CH₂—NH₂ of lysine), 1.77-1.16 (—CH₂—CH₂—CH₂—CH₂—NH— of lysine)

### Synthesis of guanidinium- and vitamin E-functionalized PLL polymer (PLL-Gua-VitE) (FIG. 1b)

PLL-Gua-VitE was prepared using a similar protocol used for synthesis of PLL-Gua. Briefly, PLL₂₂ (100 mg) was dissolved in a solution of DMSO (8.0 mL) and Et₃N (1.0 mL). Subsequently, a solution of MTC-VitE (19.6 mg) in DMSO was then added and the mixture was left to stir at RT for 8 h. After that, a solution of 1,3-bis(tert-butoxycarbonyl)-2-methyl-2-thiopseudourea (610 mg) in ACN (8.0 mL) was added and the mixture was left to stir at RT overnight. The crude PLL-Gua-VitE was then subjected to the same purification and deprotection procedures as PLL-Gua. VitE loading content was calculated by measuring the UV-Vis absorbance at 285 nm in DMSO, using the following formula:

$$VitE loading content ( \% ) = Weight of loaded Vit E / Weight of the polymer * 100 \%$$

PLL₂₂-Gua(Boc)-VitE Yield: 90%; ¹H NMR (400 MHz, CDCl₃, ppm): δ11.47 (Boc-NH—), 8.27 (—CH₂—CH₂—CH₂—CH₂—NH—), 3.86 (—CH— of backbone), 3.37 (—CH₂—NH— or —CH₂—NH₂ of lysine), 2.14-0.97 (—CH₂—CH₂—CH₂—CH₂—NH— of lysine, —CH₃ of Boc, and overlapping peaks of VitE), 0.90-0.76 (overlapping —CH₃ of VitE)

PLL₂₂-Gua-VitE Yield: 85%; ¹H NMR (400 MHz, DMSO-d, ppm): δ8.23-6.84 (—NH— and —NH₂), 4.24 (—CH— of backbone), 3.05 (—CH₂—NH— or —CH₂—NH₂ of lysine), 2.04-0.91 (—CH₂—CH₂—CH₂—CH₂—NH— of lysine and overlapping peaks of VitE), 0.89-0.75 (overlapping —CH₃ of VitE)

PLL₃₅-Gua(Boc)-VitE Yield: 91%; ¹H NMR (400 MHz, CDCl₃, ppm): δ11.46 (Boc-NH—), 8.25 (—CH₂—CH₂—CH₂—CH₂—NH—), 3.85 (—CH— of backbone), 3.36 (—CH₂—NH— or —CH₂—NH₂ of lysine), 2.16-0.95 (—CH₂—CH₂—CH₂—CH₂—NH— of lysine, —CH₃ of Boc, and overlapping peaks of VitE), 0.89-0.75 (overlapping —CH₃ of VitE)

PLL₃₅-Gua-VitE Yield: 83%; ¹H NMR (400 MHz, DMSO-d, ppm): δ8.23-6.84 (—NH— and —NH₂), 4.24 (—CH— of backbone), 3.05 (—CH₂—NH— or —CH₂—NH₂ of lysine), 2.05-0.95 (—CH₂—CH₂—CH₂—CH₂—NH— of lysine and overlapping peaks of VitE), 0.89-0.75 (overlapping —CH₃ of VitE)

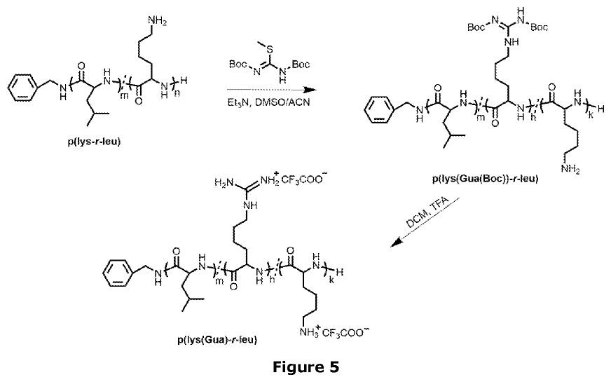

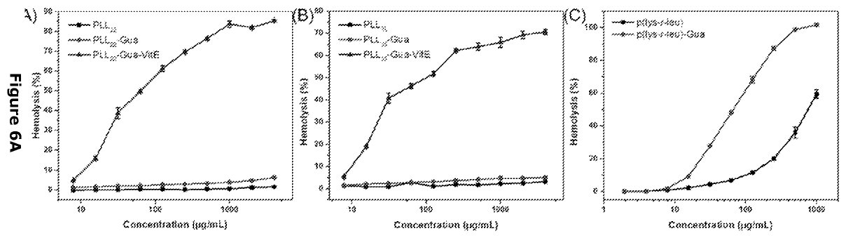

### Synthesis of guanidinium-functionalized copolymer (p(lys(Gua)-r-leu)) (FIG. 5)

Random copolymer p(lys-r-leu) was synthesized via ROP of Lys(Z)-NCA and Leu-NCA monomers with benzylamine as the initiator and a subsequent deprotection of the benzyl group. p(lys(Gua)-r-leu) was then made using a similar protocol used for synthesis of PLL-Gua.

p(lys(Z)-r-leu) Yield: 87%; ¹H NMR (400 MHz, TFA-d, ppm): δ7.35 (C₆H₅—), 5.24 (C₆H₅—CH₂— of y-benzyl group), 4.85-4.49 (—CH— of backbone), 3.27 (—CH₂—NH— of lysine), 2.13-1.33 (—CH₂—CH₂—CH₂—CH₂—NH— of lysine, —CH₂—CH— of leucine), 1.11-0.85 (—CH₃ of leucine).

p(lys-r-leu) Yield: 80%; ¹H NMR (400 MHz, D₂O, ppm): δ7.48-7.26 (C₆H₅— of initiator), 4.28 (—CH— of backbone), 2.93 (—CH₂—NH— of lysine), 1.9-1.22 (—CH₂—CH₂—CH₂—CH₂—NH— of lysine, —CH₂—CH— of leucine), 1.00-0.77 (—CH₃ of leucine).

p(lys(Gua(Boc))-r-leu) Yield: 90%; ¹H NMR (400 MHz, CDCl₃, ppm): δ11.46 (Boc-NH—), 8.27 (—CH₂—CH₂—CH₂—CH₂—NH—), 4.17-3.66 (—CH— of backbone), 3.27 and 3.06 (—CH₂—NH— or —CH₂—NH₂ of lysine), 2.16-1.16 (—CH₂—CH₂—CH₂—CH₂—NH— of lysine, —CH₂—CH— of leucine and —CH₃ of Boc), 1.00-0.72 (—CH₃ of leucine).

p(lys(Gua)-r-leu) Yield: 90%; ¹H NMR (400 MHz, D₂O, ppm): δ7.45-7.24 (C₆H₅— of initiator), 4.45-3.05 (—CH— of backbone), 3.15 and 2.99 (—CH₂—NH— or —CH₂—NH₂ of lysine), 2.01-1.18 (—CH₂—CH₂—CH₂—CH₂—NH— of lysine and —CH₂—CH— of leucine), 1.00-0.77 (—CH₃ of leucine).

### Measurement of Minimum Inhibitory Concentration (MIC) and Minimum Bactericidal Concentration (MBC)/Minimum Fungicidal Concentration (MFC)

The MICs of the polypeptides against microbes were determined using a broth micro-dilution method. The bacteria were incubated overnight in the MHB, and the bacteria suspension was diluted with MHB to achieve a concentration of 10⁸ colony-forming unit (CFU)/mL (O.D.₆₀₀=0.07). This is followed by a 1000-fold dilution in MHB. The bacterial suspension was then seeded into a 96-well plate (100 μL/well) and mixed with 100 μL of MHB containing different polypeptide concentrations. The 96-well plate was incubated for 18 h at 37° C. (bacteria) or 42 h at RT (C. albicans). The MIC was determined as the lowest concentration of the polypeptide, at which no turbidity changes were measured using a microplate reader.

The MBCs/MFCs were evaluated at the end of the MIC experiment. The microbial suspension was then spread on LB agar plates and the plates were incubated at 37° C. for 18 h (bacteria) or at RT for 42 h (yeast) for the determination of CFU on each agar plate. The experiments were performed in triplicates.

### Hemolysis Assay

The hemolysis of the polypeptides was studied using rat red blood cells (RBCs). Briefly, the whole blood was centrifuged for 10 min at 600×g and washed with fresh PBS to obtain RBCs. Rat RBCs (0.3 mL) were diluted with 10 mL of PBS for use. Polypeptides were dissolved in PBS and added into the round bottom 96-well plates (100 μL/well).

Equal volume of RBC-containing PBS solution was then added, and the plates were incubated at 37° C. for 1 h. To determine the hemolysis, the plates were centrifuged at 600×g for 10 min, 100 μL of the supernatant was transferred into a new plate. The absorbance at 576 nm was measured using a microplate reader. PBS (100 μL) was used as a negative control, while 4% Triton-X solution (100 μL) was used as a positive control. The degree of hemolysis (%) was calculated by the following formula and the data were presented as the average±SD (n=3).

$$Hemolysis ( \% ) = ( {A}_{e} - {A}_{n} ) / ( {A}_{p} - {A}_{n} ) * 100 \%$$

wherein Aₑ, Aₙ and Aₚ represent the absorbance of the experimental well, negative control well and positive control well, respectively.

### Killing Kinetics of Polypeptides

Different concentrations (1×MIC, 2×MIC, and 4×MIC) of aqueous polypeptide solutions in MHB was added to an equal volume of bacteria or yeast (C. albicans) suspension (10⁶ CFU/mL). At each designated time point, 100 μL aliquot of the suspension was removed and serially diluted in MHB, before spreading onto LB agar plates. The plates were then incubated at 37° C. for 18 h (bacteria) or at RT for 42 h (yeast) for colony counting.

### Mechanistic Study-Nitrocefin Assay

The mechanism of action was explored using nitrocefin to monitor the outer membrane permeabilization of bacteria. Briefly, the polypeptides were prepared at different concentrations in saline and 50 μL of the polypeptide solution was added into 96-well plates. The β-lactamase SHV-18 producing K. pneumoniae suspension was diluted with MHB to O.D.₆₀₀=0.11, centrifuged (3000×g) for 5 min and the resulting pellet was rinsed once with saline, before resuspending in equal volume of saline. The resulting bacteria suspension (100 μL/well) was then added to the polymer solution, followed by the addition of nitrocefin solution in saline (50 μL/well) to leave the final concentrations of the polymers at 1×MIC and 2×MIC and the nitrocefin at 50 μL/mL. The 96-well plates were incubated at 37° C. in the dark. UV absorbance at wavelength 500 nm was measured at pre-determined time points. Saline and polymyxin B (4 μL/mL) were used as negative and positive controls, respectively.

Confocal laser scanning microscopy (CLSM) Bacterial membrane permeability was also studied by propidium iodide (PI) staining and CLSM analysis. In detail, β-lactamase SHV-18 producing K. pneumoniae were incubated in a 4-well chamber slide at 37° C. overnight to allow for bacteria adhesion onto the glass slide. Polypeptide solution (2×MIC, 700 μL) was then added to each well and left to incubate at 37° C. for 1 h. The growth medium was then removed, and the wells were rinsed with PBS. The bacterial cells were stained with PI (30 μM, 1 mL) at RT for 15 min, before being rinsed with PBS (1 mL) and fixed in formalin solution for 10 min. Lastly, coverslip was placed onto the glass microscope slide and fixed with nail polish. PI staining was visualized under CLSM (Olympus FluoView™ FV300 confocal microscope).

### Evaluation of Microbial Resistance

Gram-positive MRSA was utilized as the model microbe to evaluate the development of microbial resistance against PLL₂₂-Gua. The bacteria (10⁶ CFU/agar) were streaked onto the LB agar plates, which contained PLL₂₂-Gua at different concentrations, and the plates were incubated at 37° C. overnight for the growth of colonies, if any. MBC_{agar} was defined as the concentration at which 99.9% killing was achieved. Then the MRSA of higher concentration were spread on the agar containing 1×MBC_{agar}, to determine the frequency of resistance to PLL₂₂-Gua.

### Animal

Female Balb/c mice (6-8 weeks old) and female Balb/c nude mice (6-7 weeks old) were obtained from INVIVOS PTE. LTD. (Singapore). The mice were raised in a specific pathogen free animal lab. The animal study protocols were approved by the Institutional Animal Care and Use Committee of Biological Resource Centre, Agency for Science, Technology and Research (A*STAR), Singapore.

### In Vivo MRSA-Induced Murine Wound Infection

The in vivo antibacterial efficacy of PLL₂₂-Gua was evaluated on a MRSA-infected wound infection model. In brief, hair from the back of anaesthetized mice was removed and the exposed skin was cleansed with 10% povidone-iodine solution and 75% ethanol. Three parallel wounds, with 1 cm length, were then created and infected with logarithmic growth of MRSA (1×10¹⁰ CFU/mL, 60 μL) for 1 h. Following that, one group of the mice were sacrificed to determine the bacterial count at the infection site. The remaining mice were then randomly divided into 4 groups (n=6 or 7), Group 1: saline; Group 2: PLL₂₂-Gua (10 mg/kg); Group 3: PLL₂₂-Gua (20 mg/kg); Group 4: PLL₂₂-Gua 25 (40 mg/kg). Saline or PLL₂₂-Gua solution (60 μL) was applied onto the infection site over a period of 2 days (2 dose/day). At the end of the observation period, the infected skins were collected and homogenized in 1.0 mL of sterile PBS for bacterial viability count. The results were expressed as Ig(CFU/skin). The antibacterial efficacy was quantified by reduction in bacterial counts (CFU reduction), which was calculated using the following equation:

Reduction in bacterial counts (%)=(CFU_{no treatment}−CFUₑₓₚₑᵣᵢₘₑₙₜₐₗ)/CFU_{no treatment}×100%

wherein CFU_{no treatment} and CFUₑₓₚₑᵣᵢₘₑₙₜₐₗ represent the CFU of no treatment group on Day 1 and the experimental group on Day 3, respectively. The data are presented as the average±SD (n=6 or 7).

### Acute Dermal Toxicity

Mice were randomly divided into 2 groups (3 mice/group). Hair from the back of anaesthetized mice was carefully removed to avoid injury to the skin. To increase the bio-adhesiveness of PLL₂₂-Gua, 1% (Hydroxypropyl)methyl cellulose 2910 (HPMC) was added to the polypeptide solution before application onto the skin, and the dose of PLL₂₂-Gua for each mouse was 200 mg/kg. The other group of the mice, without polypeptide treatment, served as control. The treated mice were kept under observation for 2 h to check for any abnormal behavior. After that, both groups of mice were observed for a period of 14 days, with specific attention towards changes in fur. At the end of the observation period, the body weight of the mice was recorded. Skin samples from the back and normal tissue samples (heart, liver, spleen, lung and kidney) of the mice were also collected and fixed in 10% formalin, before subjected to Hematoxylin and Eosin (H&E) staining.

### Synthesis of mPEG-b-PLG

The diblock copolymer methoxy poly(ethylene glycol)-b-poly(L-glutamic acid sodium salt) (mPEG-b-PLG) was made by ring-opening polymerization (ROP) of BLG-NCA monomer using mPEG-NH₂ as the initiator, followed by deprotection of benzyl groups.

The crude product was isolated by precipitation into an excess amount of cooled diethyl ether and dried under vacuum. It was further purified by dissolving in DMSO and saturated NaHCO₃, dialyzing against distilled water using a dialysis membrane with a molecular weight cut-off (MWCO) of 3500 Da and freeze-dried to give the pure white solid product mPEG-b-PLG. The degree of polymerization (DP) of PLG units was 35. mPEG-b-PLG Yield: 79%; ¹H NMR (400 MHz, D₂O, ppm): δ4.29 (—CH— of amide), 3.70 (PEG chain), 2.36-1.80 (—CH₂—CH₂—COONa).

### Synthesis of mPEG-b-PLL

The methoxy poly(ethylene glycol)-b-poly(L-lysine) (mPEG-b-PLL) was synthesized by ring-opening polymerization (ROP) of Lys(Z)-NCA monomer with mPEG-NH₂ as the initiator and subsequent deprotection of benzyl groups. The molecule weight of mPEG-NH₂ was 5000 Da and the degree of polymerization (DP) of PLL units was 26.

### Synthesis of mPEG-b-PLL/CDA

The methoxy poly(ethylene glycol)-b-poly(L-lysine) (mPEG-b-PLL) was synthesized to deliver PLL₂₂-Gua. The molecule weight of mPEG was 5000 Da and the degree of polymerization (DP) of PLL units was 26. To prepare the mPEG-PLL/CDA, mPEG-b-PLL (100.0 mg) was dissolved in 5% NaHCO₃, cyclohexene-1,2-dicarboxylic anhydride (CDA) (47.3 mg) was added. The reaction was stirred for 4 h at 4° C. and the mixture was dialyzed using membrane with molecular weight cut-off (MWCO) of 3500 Da against pH 9 water, and then freeze-dried to obtain the pure white solid mPEG-PLL/CDA.

mPEG-PLL/CDA Yield: 88%; ¹H NMR (400 MHz, D₂O, ppm): δ4.29 (CH of amide), 3.70 (PEG chain), 3.19 (—CH₂—CH₂—NH—CO—), 3.00 (—CH₂—CH₂—NH₂), 2.32-2.10 (—CH₂—C═C—CH₂— of CDA group), 1.62 (—CH₂—CH₂—CH₂—CH₂— of CDA group), 1.92-1.24 (—CH₂—CH₂—CH₂—CH₂—NH— and —CH₂—CH₂—CH₂—CH₂— of CDA group).

### Synthesis of quaternary ammonium-functionalized polypeptide (PLL-Qua)

Poly(L-lysine) (PLL) was synthesized and the degree of polymerization (DP) was 22. The quaternary ammonium-functioned polypeptide (PLL-Qua) was prepared through methylation reaction of PLL. Briefly, PLL (50.0 mg) was dissolved in 6.0 mL of de-ionized (DI) water/DMSO (1:1) mixture, and Et₃N (0.5 mL) was added slowly to the above solution. Iodomethane (CH₃I, 0.2 mL) was then added dropwise to the PLL solution. The reaction was left to stir in the dark at RT overnight and dialyzed using a membrane of MWCO 2000 against water, then freeze-dried to obtain white solid PLL-Qua.

Yield: 85%; ¹H NMR (400 MHz, D₂O, ppm): δ4.35 (CH of amide), 3.36 (—CH₂—CH₂—CH₂—CH₂—N—), 3.14 (—CH₃ of quaternary ammonium group), 1.97-1.24 (—CH₂—CH₂—CH₂—CH₂—N—).

### Preparation of nanoparticles (Gua-NPs, PLL-NPs, Qua-NPs)

Gua-NPs were prepared by a facile mixing method. In brief, the PLL₂₂-Gua (10.0 mg) and mPEG-PLL/CDA (18.0 mg) were separately dissolved in de-ionized water. And the mPEG-PLL/CDA solution was dropped slowly into the PLL₂₂-Gua under sonication in ice bath to obtain the nanoparticle (Gua-NPs).

PLL (4.0 mg) and mPEG-b-PLL/CDA (14.5 mg) were mixed to obtain PLL-NPs. PLL-Qua (4.0 mg) and mPEG-b-PLL/CDA (10.0 mg) were mixed to obtain Qua-NPs. In addition, the negatively charged mPEG-b-PLG was also used to prepare nanoparticles. Specifically, PLL (3.6 mg) and mPEG-b-PLG (5.0 mg) were mixed to obtain PLL-Mixture. PLL-Qua (3.6 mg) and mPEG-b-PLG (5.0 mg) were mixed to obtain Qua-Mixture. PLL-Gua (4.9 mg) and mPEG-b-PLG (5.0 mg) were mixed to obtain Gua-Mixture.

### Cell Culture

The human breast cancer cell lines BT-474, MCF-7 and MCF-7/ADR were purchased from ATCC. The cells were cultured in Roswell Park Memorial Institute 1640 medium (RPMI 1640, Gibco) supplemented with 10% fetal bovine serum (FBS) and 1% penicillin/streptomycin at 37° C. in a 5% CO₂ atmosphere. RPMI 1640 medium with pH 6.5 was prepared from RPMI 1640 by reducing its pH with HCl.

In Vitro Cytotoxicity Assays BT474 cells (7000 cells/well) were seeded in 96-well black plates and incubated overnight for cell adherence. Then, 100 μL of normal or pH 6.5 medium with different concentrations of mPEG-PLL/CDA, PLL₂₂-Gua and Gua-NPs were replaced into the plates.

After 48 h of incubation, the medium was removed, and alamarBlue cell viability reagent was added. After another 4 h of incubation in 37° C., the color change and increased fluorescence can be detected using a microplate reader (excitation: 530-560 nm; emission: 590 nm). And the cell viability was calculated according to the following equation:

$$Cell viability ( \% ) = ( {I}_{sample} - {I}_{background} ) / ( {I}_{control} - {I}_{background} )$$

Iₛₐₘₚₗₑ and I_{control} represent the fluorescent intensity of treated cells and untreated cell, respectively. I_{background} represents the fluorescent intensity of the well without cells. The data are presented as the average±SD (n=6).

In addition, the cellular cytotoxicity of mPEG-PLL/CDA, and PLL₂₂-Gua were also validated by alamarBlue assay in similar method.

In addition, the cytotoxicity of Gua-NPs and PLL₂₂-Gua against MCF-7 and MCF-7/ADR was also measured using the same assay with a cell density of 5000 cells per well.

Doxorubicin hydrochloride (DOX) was used as the positive control. For the MCF-7 and MCF-7/ADR, the initial concentration of DOX used was 10 μg/mL and 100 μg/mL, respectively.

### Killing Kinetics Against BT-474

The time-kill kinetics assay was performed using PLL-Gua and Gua-NPs against BT-474 cells. Briefly, the cells (7000/well, 100 μL/well) were seeded in 96-well black plates and incubated overnight. Then, 100 μL of medium with different concentrations of PLL-Gua (25, 50 and 100 μg/mL) and Gua-NPs (50, 100 and 150 μg/mL on PLL-Gua basis) were replaced into the wells. After incubation for a pre-determined period of time (10 min, 0.5 h, 1 h, 2 h, 4 h, 9 h 40 min, 24 h and 48 h), the media was removed, and AlamarBlue cell viability reagent was added to measure cell viability. The experiments were performed in 6 replicates, and the results were expressed as mean cell viability (%)±standard deviations shown by the error bars.

### Synthesis of Alexa Fluor 488-labelled Gua-NPs (AF-488@Gua-NPs)

Alexa Fluor™ 488 NHS ester (1.0 mg) was dissolved in 2 mL of DMSO, and PLL-Gua (19 mg) was dissolved in 5 mL of 0.1 M NaHCO₃. The two solutions were mixed and stirred at RT for 4 h. The mixture was dialyzed using a membrane of MWCO 1000 against DI water to remove free dye molecules, and then freeze-dried to obtain yellow-green solid Alexa Fluor 488-labelled PLL-Gua (AF-488@PLL-Gua). AF-488@PLL-Gua (5.0 mg) and mPEG-b-PLL/CDA (9.0 mg) were separately dissolved in DI water. mPEG-b-PLL/CDA solution was added dropwise into PLL-Gua solution under sonication in an ice bath to obtain Alexa Fluor 488-labelled Gua-NPs (AF-488@Gua-NPs). The formation and stability of Gua-NPs were measured in different pH buffer.

Mechanistic study of Gua-NPs

The anticancer mechanism of Gua-NPs was explored by flow cytometry and confocal laser scanning microscopy (CLSM). For the flow cytometry assay, MCF-7 cells (2×10⁵/well) were seeded in 12-well plates and incubated overnight. The media was replaced with fresh medium containing Gua-NPs (50 and 100 μg/mL on PLL-Gua basis) or PLL-Gua (25 and 50 μg/mL), and incubated for 2 h. Paclitaxel (PTX, 48 μg/mL) and Triton-X 100 (0.01%) were used as controls to induce apoptosis and necrosis, respectively. After 2 h of incubation, the cells were detached and trypsinized, collected and centrifuged at 1500 rpm for 5 mins. Next, the cells were stained with Alexa Fluor® 488 annexin V and PI according to the manufacturer's instructions. The samples were then analyzed by BD LSR II flow cytometry (BD Biosciences, U.S.A.). The experiments were performed in 3 replicates, and the results were expressed as mean apoptosis ratio (%)±standard deviations shown by the error bars.

For the CLSM assay, BT-474 cells (1.4×10⁵/chamber, 0.7 mL/chamber) were seeded in the chamber (Nunc® Lab-Tek® II chambered coverglass, borosilicate glass, chamber size 4) and incubated overnight. Next, fresh medium containing Alexa Fluor 488-labelled Gua-NPs (25 μg/mL on Alexa Fluor 488-labelled PLL-Gua basis) or Alexa Fluor 488-labelled PLL-Gua (25 μg/mL) were added and incubated at 37° C. for 30 min. Then, the cells were washed with phosphate-buffered saline (PBS) before Hoechst 33342 solution (2 μM) was added and incubated at 37° C. for 10 min. After washing the residual Hoechst 33342 with PBS, LysoTracker™ Deep Red solution (100 nM) was added and incubated for 10 min at 37° C. Finally, the samples were visualized using a CLSM (Carl Zeiss LSM 710 confocal microscope, Germany).

### Inhibition of Cell Migration

MCF-7 cells (8×10⁵/well) were seeded in 6-well plates and incubated overnight for cell adhesion. To create a scratch, a 200 μL pipet tip was used to scrape a straight line through the cell layer. The old media was removed and washed with PBS, and the cells observed under a microscope to acquire images of the original scratch. Fresh medium containing DOX, PLL-Gua or Gua-NPs at IC50 (a polymer concentration that led to 50% inhibition of cell growth) was added and incubated for 24 h. After that, the scratches were observed and imaged using a microscope (Leica, Germany).

### Cellular Morphology Staining

MCF-7 and MCF-7/ADR cells were seeded at a density of 1000 cells/well in 50 μL medium, in a 384-well plate (PerkinElmer View Plate-384 Black). The plate was incubated overnight at 37° C., 5% CO₂ for cell recovery. Treatment was performed at final concentrations of 25 μg/mL for PLL-Gua and Gua-NPs (on PLL-Gua basis) and 5.8 μg/mL for DOX, with a final volume of 40 μL per well. Cells were incubated with PLL-Gua, Gua-NPs or DOX for 24 h (37° C., 5% CO₂). Subsequently, mitochondria were stained with Mito Tracker Deep Red solution in medium, with a final concentration of 100 nM. The cells were incubated in the dark at 37° C. with 5% CO₂ for 30 min. To fix the cells, 20 μL of 16% formaldehyde in PBS were added to each well (final concentration: 4%) and incubated in the dark at room temperature for 20 min. The cells were washed two times with 70 μL of Hanks' balanced salt solution (HBSS). To each well, 20 μL of staining solution was added, which contains 1% bovine serum albumin (BSA), 0.1% Triton X-100, 8.25 nM Alexa Fluor™ 594 Phalloidin, 5 μg/mL Concanavalin A (Alexa Fluor™ 488 conjugate), 1 μg/mL Hoechst 33342, 1.5 μg/mL Wheat-Germ Agglutinin-Alexa555 conjugate and 6 μM SYTO™ 14 solution. The cells were incubated in the dark at room temperature for 30 min. Then, the cells were washed three times with 70 μL HBSS. The HBSS was not aspirated after the final washing step. The plates were sealed and centrifuged for 1 min at 500 rpm. The plate was then imaged using an Opera Phenix high content screening microscope (PerkinElmer) in 4 channels (Ex405/Em456; Ex488/Em522; Ex561/Em599; Ex640/Em706) with 9 sites per well and 63× magnification (binning 2).

### Evaluation of In Vivo Toxicity

Fifteen healthy Balb/c mice were randomly divided into 3 groups: Group 1: untreated control group (n=5); Group 2: PLL-Gua (10 mg/kg, n=5); Group 3: Gua-NPs (20 mg/kg on PLL-Gua basis, n=5). The PLLGua and Gua-NPs were injected via tail veil on Day 0, 4, 8, 11, 15 and 18. All mice were sacrificed on Day 21. Blood samples were collected for blood chemistry and blood routine assays. Major organs (heart, liver, spleen, lungs and kidneys) were excised and fixed in formalin solution, embedded in paraffin, and then stained with hematoxylin and eosin (H&E).

### Evaluation of LD50

LD50 is the dose that causes death in 50% of the treated mice during a given period.

In brief, the Balb/c mice were injected intravenously (i.v.) with PLL-Gua or Gua-NPs at different doses. LD50 values was estimated from the survival rate of treated mice over 7 days using the Dixon's method (J. Am. Statistical Association 60 (1965) 967-978).

### In Vivo Biodistribution

BT-474 cells (5×10⁶/mouse) suspended in Matrigel/RPMI 1640 medium (1:1) were injected subcutaneously into the right flank of the Balb/c nude mice. Meanwhile, 17β-estradiol pellets (1 pellet/mice) were inoculated subcutaneously into the neck of mice to induce tumor growth. After 26 days, the mice bearing BT-474 tumors were divided into 2 groups: Group 1: Alexa Fluor 750-labelled PLL-Gua (10 mg/kg); Group 2: Alexa Fluor 750-labelled Gua-NPs (10 mg/kg on Alexa Fluor 750-labelled PLL-Gua basis). At pre-determined time points (6, 24 and 48 h), the mice (n=3/group) were sacrificed, and tumors and normal tissues (brain, heart, liver, spleen, lungs and kidneys) were excised and imaged using a small animal IVIS in vivo imaging system (PerkinElmer, U.S.A.).

### Anticancer Efficacy In Vivo

BT474 cells (5×10⁶/mouse) suspended in Matrigel/RPMI 1640 medium (1:1) were injected subcutaneously into the right flank of the Balb/c nude mice. Meanwhile, the 17β-estradiol pellets (1 pellet/mice) were inoculated subcutaneously into the neck of mice to induce tumor growth. After 21 days, the mice bearing BT474 tumors (˜130 mm³) were divided into 2 groups (Day 0): Group 1: untreated control; Group 2: Gua-NPs (20 mg/kg PLL₂₂-Gua). Gua-NPs were injected via tail veil on Day 0, 2, 7, 9, 14 and 17. Tumor volume was measured by a Vernier caliper to evaluate the antitumor activity.

Body weight was also recorded to assess the systemic toxicity of Gua-NPs. At Day 21, the mice were sacrificed and the excised tumors were photographed and weighed. The tumor volume (Vₜ, mm³) and tumor suppression rates (TSR, %) were calculated via the following formulas:

$$\begin{matrix}Tumor volume ( {V}_{t} , {mm}^{3} ) = a \times {b}^{2} / 2 \\ Tumor suppression rates ( TSR , \% ) = \{ ( {V}_{c} - {V}_{e} ) / {V}_{c} \} \times 100 \%\end{matrix}$$

where a and b were the longest and shortest diameter of tumor, respectively. V_{c} and Vₑ were the average of tumor volume of control group and experimental group on Day 21, respectively.

### Histological Analysis of Tumor Tissues and Normal Organs of Tumor Bearing Mice

On Day 21, the tumors and major organs (heart, liver, spleen, lungs and kidneys) were excised and fixed in formalin solution, embedded in paraffin, and then stained with H&E. The tumors were also stained with terminal deoxynuceotidyl transferase-mediated dUTP nick-end labelling (TUNEL) (Merck, U.S.A.) according to the manufacturer's instruction.

### Statistical Analyses

Data are expressed as mean±standard deviation (SD). Data were analyzed for statistical significance using two-tailed Student's t-test. *p<0.05, **p<0.01, and ***p<0.001 were considered statistically, highly, and extremely significant difference, respectively.

### Synthesis of PLL-Gua

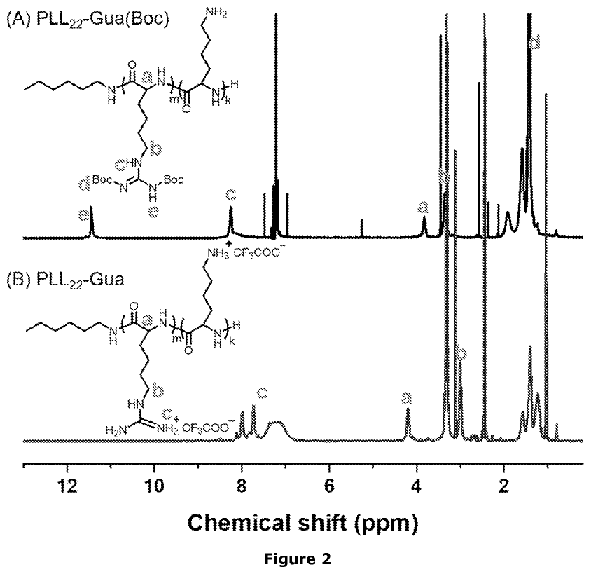

PLL with DP of 22 and 35 were synthesized and denoted as PLL₂₂ and PLL₃₅, respectively. Guanidinium-functionalized PLL was synthesized using 1,3-bis(tert-butoxycarbonyl)-2-methyl-2-thiopseudourea as the guanidylating agent, followed by the TFA-mediated deprotection of the Boc protecting group (FIG. 1A). Using PLL₂₂-Gua as a representative example, the success of guanidinium-functionalization was confirmed by the appearance of peaks d (—CH₃ in Boc group) and e (Boc-NH—) at 5 1.46 and 11.46 ppm, respectively (FIG. 2A). The degree of functionalization, determined by the integration area under peaks a and e, was 95%. The disappearance of peak e and d (FIG. 2B) indicates complete deprotection of the Boc protecting group. GPC analysis revealed that PLL₂₂-Gua and PLL₃₅-Gua had a number average molecular weight (Mn) of 3.3×10³ and 4.7×10³ g/mol, respectively. Furthermore, they had a narrow distribution with PDI of 1.4 and 1.3, respectively.

### Synthesis of PLL-Gua-VitE

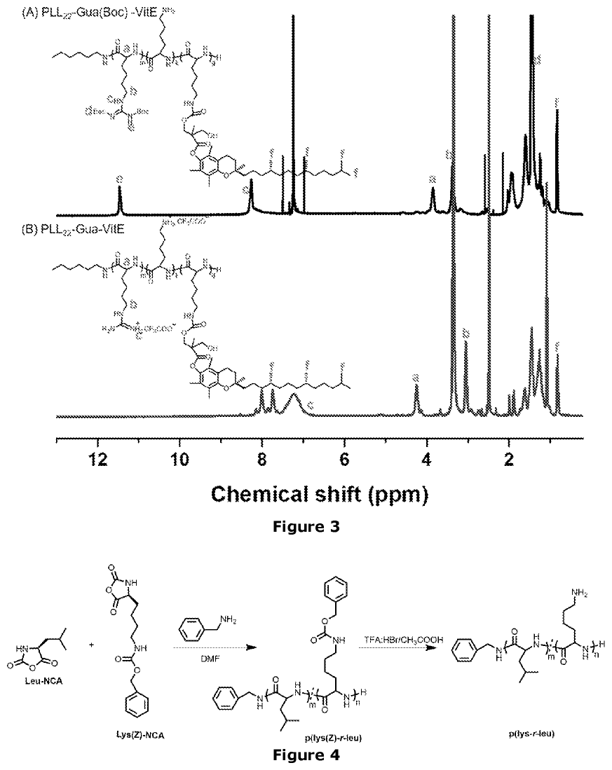

Polymer hydrophobicity affects its antimicrobial activity. To increase the hydrophobicity of the PLL-Gua polymer, a vitamin E group was conjugated via the ring-opening of vitamin E-functionalized cyclic carbonate (MTC-VitE). The synthesis of PLL-Gua-VitE was similar to that of PLL-Gua, and the vitamin E group was conjugated onto the side chain (FIG. 1B). Using PLL₂₂-Gua(Boc)-VitE as a representative example, the appearance of peak f (—CH₃ in vitamin E), at 5 0.90-0.76 ppm, indicated the successful conjugation of vitamin E (FIG. 3). The subsequent disappearance of peaks e and d confirmed the complete deprotection of the Boc groups. The vitamin E content of PLL₂₂-Gua-VitE and PLL₃₅-Gua-VitE was determined by UV-Vis spectroscopy, and found to be 15.9% and 9.2%, respectively.

With an additional hydrophobic component, PLL₂₂-Gua-VitE and PLL₃₅-Gua-VitE were capable of self-assembly, forming micelles with diameters of 16 nm and 13 nm, respectively. Additionally, their low critical micelle concentration (CMC) values (8.3 and 11.1 μg/mL, respectively) suggest that these micelles have excellent stability. With zeta potential (Q of +8.6+0.98 and +11.3+1.18 mV in PBS, respectively, these micelles are capable of interacting with the negatively charged surface of bacterial membrane.

### Synthesis of p(lys(Gua)-r-leu)

Amphiphilic random copolymer p(lys-r-leu), containing both amine functional group and leucine group, was also synthesized via the ROP of Lys(Z)-NCA and Leu-NCA using benzylamine as the initiator, followed by the deprotection of the benzyl group in TFA and HBr/CH₃COOH (FIG. 4). The successful synthesis of p(lys-r-leu) was confirmed, by ¹H NMR spectroscopy, which contains 29 repeating units of lysine and 12 repeating units of leucine. In ¹H NMR, the disappearance of peaks e (-PhH-) and d (Ph-CH₂—) at δ 7.35 and 5.25 ppm, respectively, indicated successful deprotection of P(lys(Z)-r-leu). GPC analysis of p(lys-r-leu) revealed that the polypeptide had a Mn of 5.1×10³ g/mol and a narrow distribution with PDI of 1.1.

The guanidinium-functionalization of p(lys-r-leu) was done in a similar fashion as for PLL (FIG. 5), as confirmed by the appearance of peaks d (—CH₃ in Boc group) and e (Boc-NH—) at 6 1.45 and 11.46 ppm, respectively, with a degree of functionalization of 93%. The complete Boc-deprotection in DCM/TFA was confirmed by the disappearance of peaks d and e. The resulting polypeptide p(lys(Gua)-r-leu) had a Mn of 4.5×10³ g/mol and a narrow distribution, with PDI of 1.2. Interestingly, p(lys-r-leu) and p(lys(Gua)-r-leu) did not form micelles, even at the concentration of 1000 μg/mL.

### In Vitro Antimicrobial Activity

The in vitro antimicrobial activity of the synthesized guanidinium-functionalized polypeptides was evaluated against a range of microbes, including Gram-positive bacteria (S. aureus and MRSA), Gram-negative bacteria (E. coli, A. baumannii, P. aeruginosa and K. pneumoniae) and yeast C. albicans. The results are summarized in Table 1. All guanidinium-functionalized polypeptides exhibited broad-spectrum antimicrobial activity with MIC of 15.6-125 μg/mL. The guanidinium-functionalized PLL polymers were more effective as compared to their non-functionalized counterparts, especially against S. aureus, MRSA, A. baumannii, K. pneumonia and C. albicans. This highlights the importance of the guanidinium functionalization. However, increasing the hydrophobicity of PLL-Gua, through the addition of vitamin E group in its side chain, offered no significant enhancement in antimicrobial activity, but it led to reduced bactericidal activity against S. aureus, MRSA, K. pneumonia and C. albicans probably because the micelle formation of PLL-Gua-VitE prevented its hydrophobic component from interacting with bacterial membrane. MBC values of p(lys(Gua-r-leu) with greater hydrophobicity were lower than those PLL₃₅-Gua against E. coli and A. baumannii (Table 1). Interaction of the hydrophobic VitE with proteins present in the growth medium might prevent it from inserting into bacterial membrane. In addition, the length of the polypeptides did not appear to impact their antimicrobial activity. The (MBC or MFC)/MIC ratio (R) of antimicrobial agents could be used to evaluate their bactericidal/fungicidal potential. A polymer is of good bactericidal/fungicidal potential if their R values do not exceed 4. The R values of the guanidinium-functionalized polypeptides (PLL-Gua, p(lys(Gua-r-leu)) were less than 4 against most bacterial types tested. As such, PLL-Gua and p(lys(Gua-r-leu) are good candidates for treatment of bacterial infection in vivo.

TABLE 1
MIC and MBC(μg/mL) of synthesized antimicrobial agents against various types
of microbes. R = (MBC or MFC)/MIC. R ≤ 4 signifies bactericidal/fungicidal activity.

| | Gram-positive | | | | | | Gram-negative | | | | | |
|---|---|---|---|---|---|---|---|---|---|---|---|---|
| | S. aureus | | | MRSA | | | E. coli | | | P. aeruginosa | | |
| Polymers | MIC | MBC | R | MIC | MBC | R | MIC | MBC | R | MIC | MBC | R |
| PLL₁₁ | 15.6 | 125 | | | | | 31.25 | 62.5 | | 125 | 1000 | |
| PLL₂₂ | 15.6 | 62.5 | 4 | 31.3 | 125 | 4 | 31.3 | 31.3 | 1 | 62.5 | 125 | 2 |
| PLL₃₅ | 15.6 | 31.3 | 2 | 15.6 | 250 | 16 | 15.6 | 15.6 | 1 | 62.5 | 125 | 2 |
| PLL₂₂-Gua | 15.6 | 15.6 | | | | | 31.25 | 31.25 | | 125 | 1000 | |
| PLL₂₂-Gua | 15.6 | 15.6 | 1 | 15.6 | 15.6 | 1 | 15.6 | 500 | 32 | 62.5 | 500 | 8 |
| PLL₃₅-Gua | 15.6 | 15.6 | 1 | 15.6 | 31.3 | 2 | 31.3 | 250 | 8 | 62.5 | 125 | 2 |
| PLL₂₂-Gua- | 15.6 | 62.5 | | | | | 62.5 | 1000 | | 125 | 500 | |
| VitE | | | | | | | | | | | | |
| PLL₂₂-Gua- | 31.3 | 125 | 4 | 31.3 | 62.5 | 2 | 62.5 | 500 | 8 | 125 | 500 | 4 |
| VitE | | | | | | | | | | | | |
| PLL₃₅-Gua- | 31.3 | 125 | 4 | 31.3 | 62.5 | 2 | 62.5 | 500 | 8 | 125 | 1000 | 8 |
| VitE | | | | | | | | | | | | |
| p(lys-r-leu) | 7.8 | 15.6 | 2 | 31.3 | >1000 | >32 | 125 | 1000 | 8 | 15.6 | 250 | 16 |
| p(lys(Gua- | 15.6 | 62.5 | 4 | 15.6 | 31.3 | 2 | 15.6 | 31.3 | 2 | 31.3 | 125 | 32 |
| r-leu) | | | | | | | | | | | | |

| | Gram-negative | | | | | | Yeast | | |
|---|---|---|---|---|---|---|---|---|---|
| | A. baumannii | | | K. pneumoniae | | | C. albicans | | |
| Polymers | MIC | MBC | R | MIC | MBC | R | MIC | MBC | R |
| PLL₁₁ | >1000 | >1000 | | >1000 | >1000 | | 62.5 | 125 | |
| PLL₂₂ | >1000 | >1000 | — | 250 | >1000 | >4 | 250 | 500 | 2 |
| PLL₃₅ | 1000 | 1000 | 1 | 125 | 1000 | 8 | 500 | 500 | 1 |
| PLL₂₂-Gua | 125 | 250 | | 125 | 500 | | 31.25 | 62.5 | |
| PLL₂₂-Gua | 62.5 | 62.5 | 1 | 62.5 | 500 | 8 | 31.3 | 31.3 | 1 |
| PLL₃₅-Gua | 62.5 | 250 | 4 | 62.5 | 500 | 8 | 31.3 | 31.3 | 1 |
| PLL₂₂-Gua- | 125 | 500 | | 250 | >1000 | | 31.25 | 500 | |
| VitE | | | | | | | | | |
| PLL₂₂-Gua- | 125 | 250 | 2 | 125 | >1000 | >8 | 31.5 | 62.5 | 2 |
| VitE | | | | | | | | | |
| PLL₃₅-Gua- | 125 | 125 | 1 | 125 | >1000 | >8 | 31.3 | 62.5 | 2 |
| VitE | | | | | | | | | |
| p(lys-r-leu) | 31.3 | 62.5 | 2 | 500 | >1000 | >2 | 62.5 | >1000 | >16 |
| p(lys(Gua- | 31.3 | 62.5 | 2 | 62.5 | >1000 | >16 | 15.6 | 1000 | 64 |
| r-leu) | | | | | | | | | |

### Hemolytic Activity of the Polypeptides

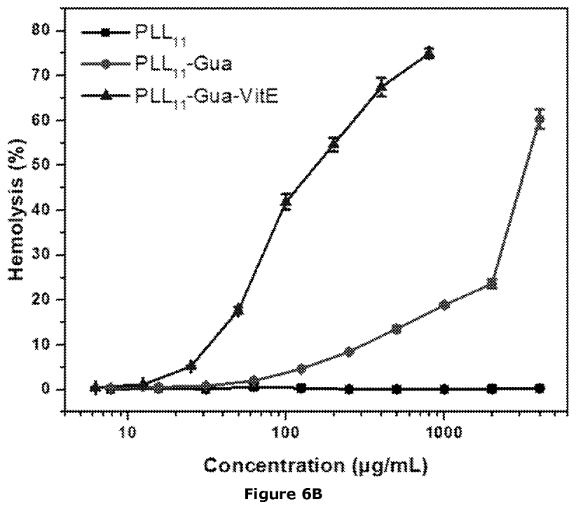

Hemolytic activity has been widely used as an initial metric of toxicity for membrane-active antibacterial polymers. As shown in FIG. 6A, 6B and Table 2, PLL₂₂-Gua and PLL₃₅-Gua remained non-hemolytic (HC₅₀>4000 μg/mL) after guanidinium-functionalization. Their low hemolytic activity indicated the high selectivity of PLL-Gua towards microbes over mammalian cells. It was also observed that the introduction of hydrophobic vitamin E and leucine increased the hemolytic activity of the polypeptides. The functionalization of p(lys-r-leu) with guanidinium functional groups also led to an increase in its hemolytic activity.

TABLE 2
Hemolysis assay against rat RBCs in vitro.

| | Entry | ^{a}HC₅₀(μg/mL) | ^{a}HC₁₀(μg/mL) |
|---|---|---|---|
| Hydrophilic | PLL₁₁ | >4000 | >4000 |
| polymer | PLL₂₂ | >4000 | >4000 |
| | PLL₃₅ | >4000 | >4000 |
| | PLL₁₁-Gua | 4000 | 500 |
| | PLL₂₂-Gua | >4000 | >4000 |
| | PLL₃₅-Gua | >4000 | >4000 |
| Amphiphilic | PLL₁₁-Gua-VitE | 200 | 50 |
| polymer | PLL₂₂-Gua-VitE | 125 | 15.6 |
| | PLL₃₅-Gua-VitE | 125 | 15.6 |
| Random | p(lys-r-leu) | 1000 | 125 |
| polymer | p(lys(Gua)-r-leu) | 125 | 15.6 |

^{a}HC₅₀ or HC₁₀ was the minimum concentration at which at least 10% or 50% of the maximum lysis was observed in the hemolysis assays.

### Killing Kinetics of Polypeptides

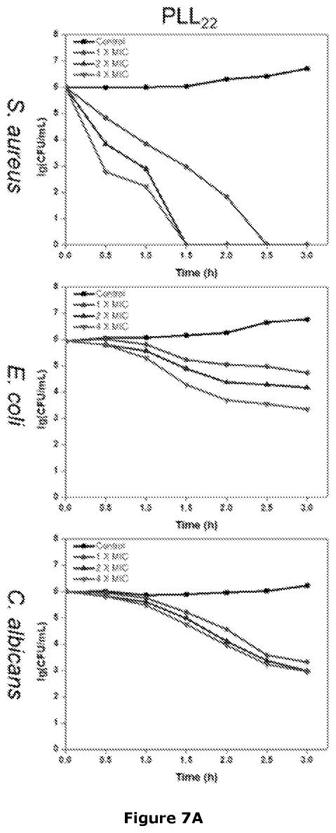

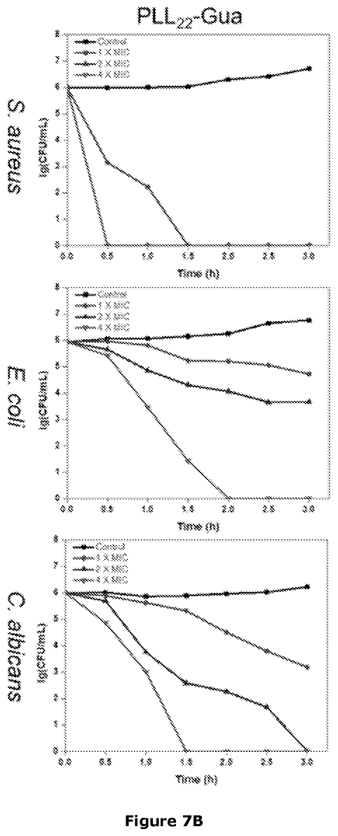

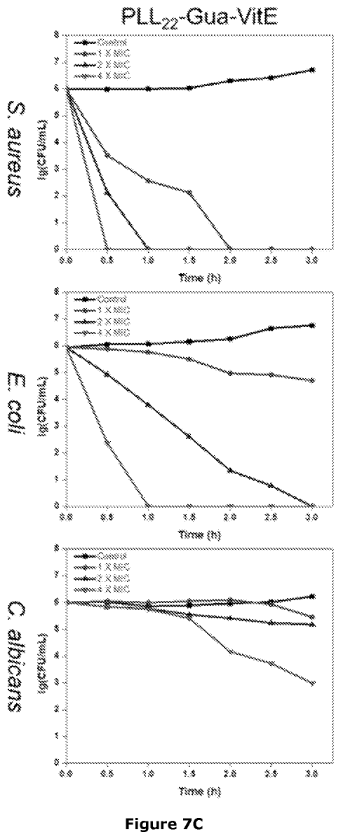

The killing kinetics of PLL₂₂, PLL₂₂-Gua and PLL₂₂-Gua-VitE against S. aureus, E. coli and C. albicans at 1×MIC, 2×MIC and 4×MIC was examined in vitro. Generally, the polypeptides demonstrated fast killing kinetics, with the rate of bacterial eradication being concentration-dependent (FIG. 7). These polypeptides killed S. aureus much faster than E. coli and C. albicans at the same concentration. For example, while PLL₂₂-Gua, at 2×MIC, was able to eradicate S. aureus completely within 0.5 h, it had to take 3 h to completely eradicate C. albicans and even after 3 h, it had only resulted in a 2-log reduction for E. coli. Comparing PLL₂₂-Gua and PLL₂₂, it is evident that the presence of guanidinium-functionalized group enhanced killing kinetics. PLL₂₂-Gua exhibited the fastest killing kinetics against S. aureus at 1×MIC and 2×MIC. At 1×MIC, PLL₂₂-Gua required 1.5 h while PLL₂₂ and PLL₂₂-Gua-VitE took 2.5 and 2.0 h, respectively, to achieve a 6-log reduction in bacteria count. Collectively, PLL₂₂-Gua presented itself as an excellent antimicrobial candidate.

### Mechanistic Study-Nitrocefin Assay

Nitrocefin, containing a β-lactam ring, is susceptible to β-lactamase and undergoes a rapid color change from yellow to red upon hydrolysis. SHV-18, a β-lactamase produced by K. pneumoniae, resides in the periplasmic space. Therefore, a compromised bacterial outer membrane would lead to the hydrolysis of nitrocefin since the compromised membrane allows nitrocefin to permeate. Polymyxin B, capable of disrupting the outer bacterial membrane, was used as the positive control, whereas saline was served as the negative control.

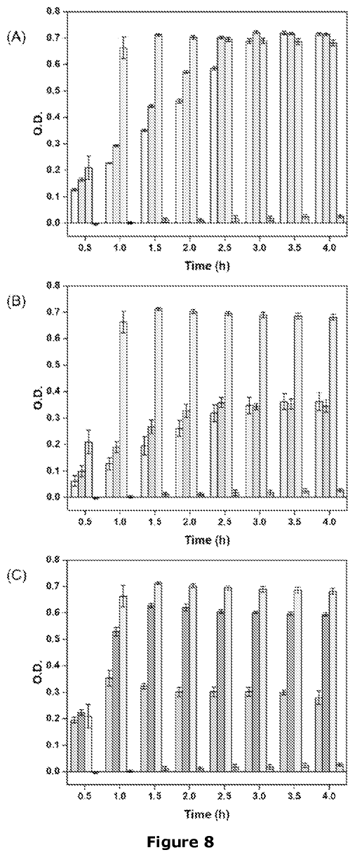

As shown in FIG. 8A, PLL₂₂ at 1×MIC and 2×MIC caused the same extent of nitrocefin hydrolysis as polymyxin B at 3 h post incubation. This indicated that PLL₂₂ killed bacteria by disrupting their outer membrane and the extent of disruption was time-dependent. On the other hand, PLL₂₂-Gua only caused ˜50% nitrocefin hydrolysis relative to polymyxin B, even after 4 h of incubation (FIG. 8B). This suggests that PLL₂₂-Gua killed bacteria mainly by membrane translocation. This explains why an increase in the concentration of PLL₂₂-Gua from 1×MIC to 2×MIC did not result in an increase in nitrocefin hydrolysis. Interestingly, nitrocefin cleavage caused by PLL₂₂-Gua-VitE was concentration-dependent (FIG. 8C). This implied that PLL₂₂-Gua-VitE disrupted the outer bacterial membrane at a higher concentration (2×MIC) probably due to enhanced hydrophobic interaction between the polypeptide and the lipid domain of the membrane.

### Mechanistic Study-CLSM Analysis

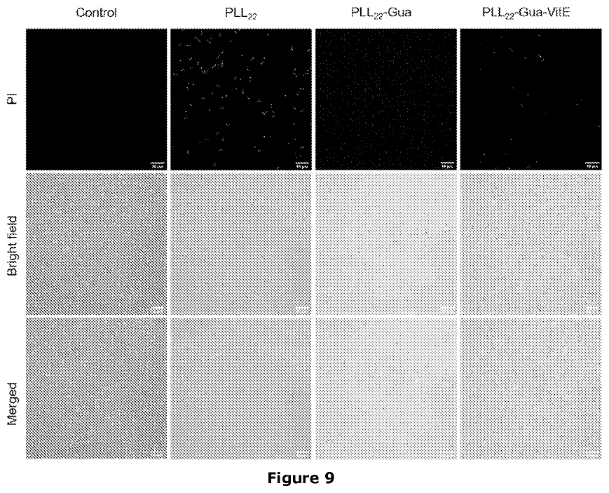

The changes in membrane permeability could also be investigated by CLSM using PI staining. If the membrane integrity is compromised, PI will be able to penetrate the cell and bind to bacterial DNA, resulting in enhanced fluorescence intensity. As shown in FIG. 9, red fluorescence was not observed in the saline-treated K. pneumoniae, implying that their bacterial membrane was intact. In contrast, the red fluorescence was observed in most bacterial cells, which were treated with PLL₂₂ for 1 h, indicating that their membrane integrity was compromised. This membrane-disruptive behavior was also observed in other synthetic antimicrobial polymers containing quaternary ammonium groups.

However, bacteria treated with PLL₂₂-Gua showed little red fluorescence, signifying that PLL₂₂-Gua killed bacteria via membrane translocation mechanism. On the other hand, red fluorescence was observed in part of bacterial cells after incubation with PLL₂₂-Gua-VitE for 1 h, indicating the polymer caused membrane disruption to some extent. These observations are consistent with results from the nitrocefin staining assay.

### Evaluation of Bacterial Resistance

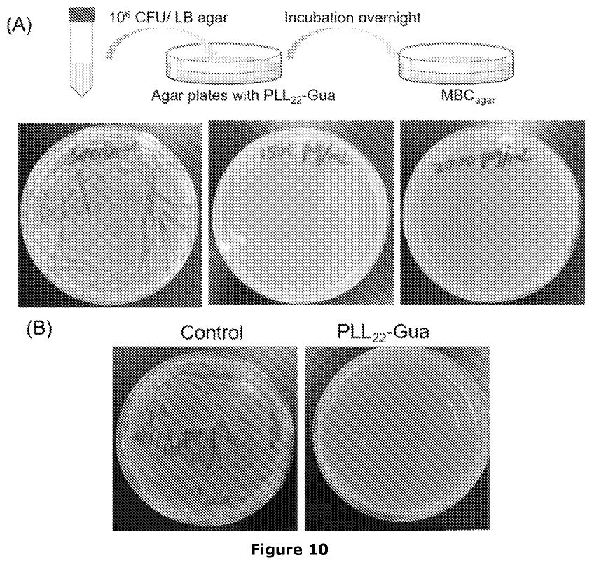

Antibiotic-resistant “superbugs” were predicated to cause more than 10 million death per year by 2050. Therefore, it is important to evaluate the frequency of resistance to PLL₂₂-Gua against bacteria. MRSA (10⁶ CFU/agar) was streaked on LB agar plates, containing various concentrations of PLL₂₂-Gua, to determine the MBC_{agar}, where 99.9% bacteria killing was achieved. As shown in FIG. 10A, the MBC_{agar} of PLL₂₂-Gua was 1500 μg/mL. After that, MRSA suspension (10⁷ CFU/agar) was then streaked onto a LB agar plates containing 1500 μg/mL of PLL₂₂-Gua (FIG. 10B). There was no resistant colony on the plates, so the frequency of resistance to PLL₂₂-Gua was determined to be less than 1/(3×10⁷)=3.3×10⁻⁸. The low resistant frequency demonstrated the potential application of PLL₂₂-Gua as a macromolecular antimicrobial agent to combat antimicrobial resistance in vivo.

### In Vivo Antimicrobial Efficacy in a MRSA-Induced Murine Wound Infection Model

To demonstrate the potential application of PLL₂₂-Gua in treating MDR infection in vivo, PLL₂₂-Gua was applied to treat MRSA-induced wound infection in a murine model. MRSA (6×10⁸ CFU/skin) was applied to the wound sites for 1 h, followed by a 4-dose treatment of PLL₂₂-Gua solution over a period of 2 days (2 doses/day). To assess the in vivo bactericidal effect of PLL₂₂-Gua, the infected skin tissues were collected, homogenized, and cultured on the LB agar plates for CFU counting (FIG. 11A). In the saline-treated group, the bacterial load decreased by 0.71 Ig CFU on Day 3, indicative of a stable bacterial burden. Conversely, the treatment with PLL₂₂-Gua resulted in substantially higher log-reduction of 2.07 Ig CFU, 2.26 Ig CFU, and 2.30 Ig CFU with doses of 10 mg/kg, 20 mg/kg, and 30 mg/kg, respectively (FIG. 11A). The percentage of bacteria removal was 99.1%, 99.4% and 99.5% when PLL₂₂-Gua was applied at doses of 10 mg/kg, 20 mg/kg, and 30 mg/kg, respectively, demonstrating excellent in vivo antimicrobial efficacy. Given the in vivo validation, PLL₂₂-Gua is a promising macromolecular antimicrobial agent in combating the MRSA-caused skin infection.

### Acute Dermal Toxicity

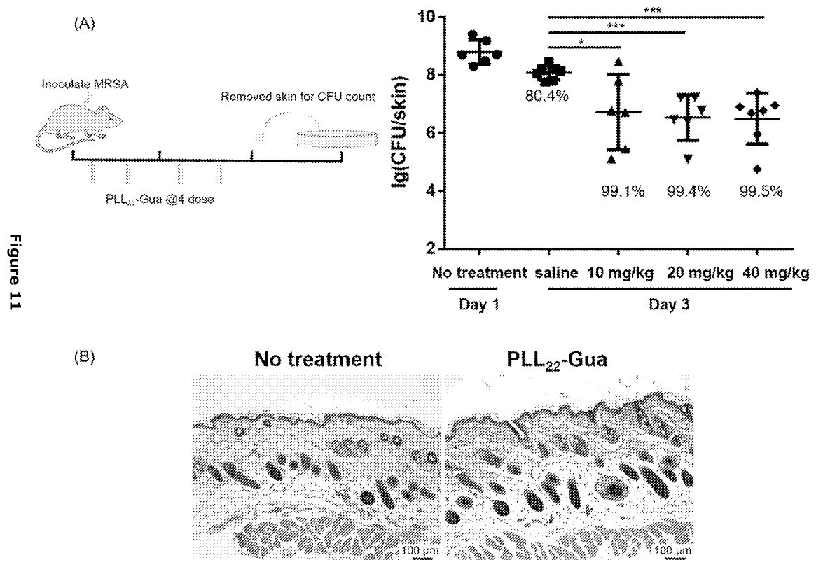

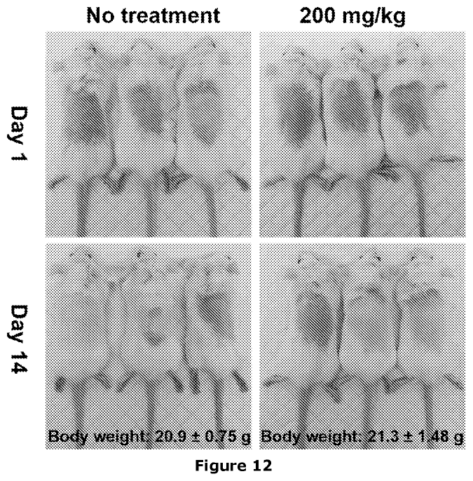

Acute dermal toxicity is another important metric, especially for dermally administered substances. In this assay, a solution of PLL₂₂-Gua, mixed with 1% HPMC for bio-adhesive improvement, was applied onto the skin of the mice. The dose of PLL₂₂-Gua used was 200 mg/kg, which is much higher than the effective dose required for treatment. The mice were then observed over a period of 14 days, for any abnormalities, as recommended by the Organization for Economic Cooperation and Development (OECD) guidelines. During the period of observation, the mice showed no abnormalities in their skin, fur, eyes, and respiratory system. In addition, they did not exhibit any abnormal behavior traits. The PLL₂₂-Gua-treated mice also showed no significant difference in their body weight, as compared with the control group. Moreover, almost complete fur regeneration was observed in both groups of mice (FIG. 12), indicating the non-toxic nature of PLL₂₂-Gua. H&E staining of the skin (FIG. 11B) and tissue (FIG. 13) samples showed complete and normal structures, without any significant pathological abnormalities. This proves that the use of PLL₂₂-Gua is safe and promising for the treatment of MRSA-induced wound infection.

### Preparation and characterization of drug-free anticancer polypeptide nanoparticles and Synthesis of mPEG-b-PLL/CDA and Preparation of nanoparticles Gua-NPs

Cationic polypeptides with three different charge groups were synthesized. Poly(L-lysine) (PLL) and guanidiniumfunctionalized PLL (PLL-Gua) with DP of 22 were synthesized as disclosed above. Quaternary ammonium functionalized PLL (PLL-Qua) was prepared through methylation reaction of PLL with iodomethane. The peak c (6 3.14, —CH₃ of quaternary ammonium group) indicated the successful functionalization, and the functionalization degree was determined to be 95%.

Next, two negatively charged polypeptides were synthesized. The simple anionic block polypeptide, methoxy poly(ethylene glycol)-b-poly(L-glutamic acid sodium salt) (mPEG-b-PLG), had DP of 35.

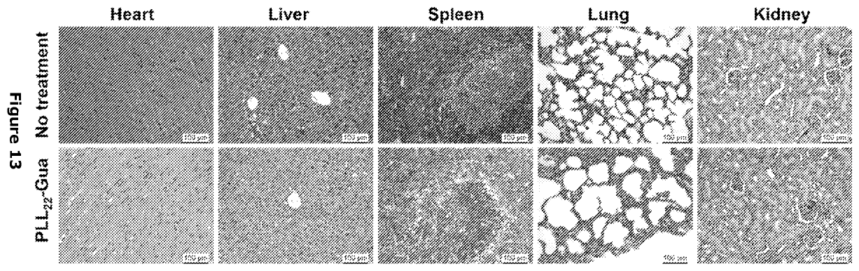

pH-sensitive anionic polymer mPEG-PLL/CDA was synthesized through reaction between primary amines of methoxy poly(ethylene glycol)-b-poly(L-lysine) (mPEG-b-PLL) and cyclohexene-1,2-dicarboxylic anhydride (CDA) (FIG. 14A). As shown in FIG. 14B, the conjugation degree of CDA was 77% (m=6, n=20), determined by comparing the peak c and c′. The ¹H NMR results showed that mPEG-PLL/CDA was pH-sensitive, and the lower pH induced higher (greater and faster) hydrolysis of CDA (FIG. 14C).

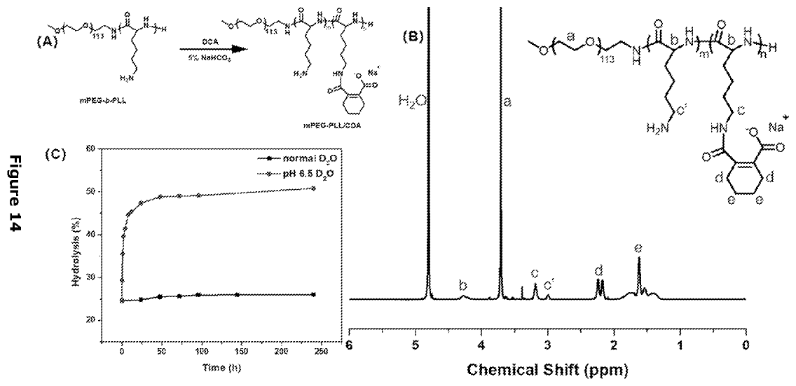

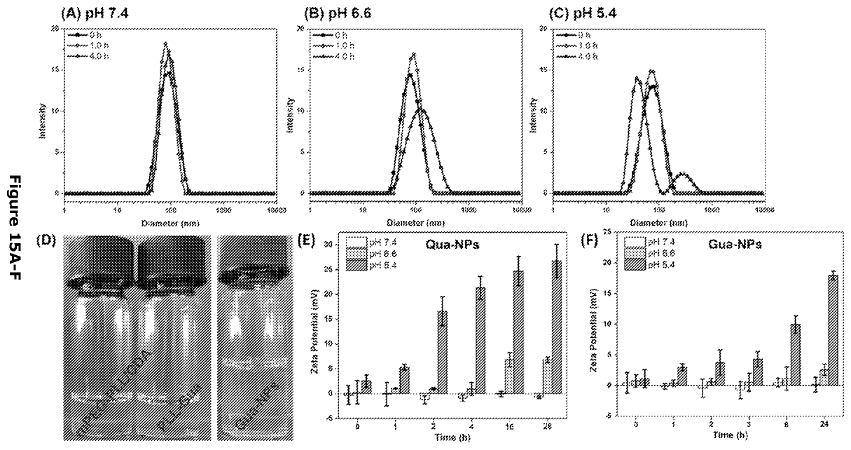

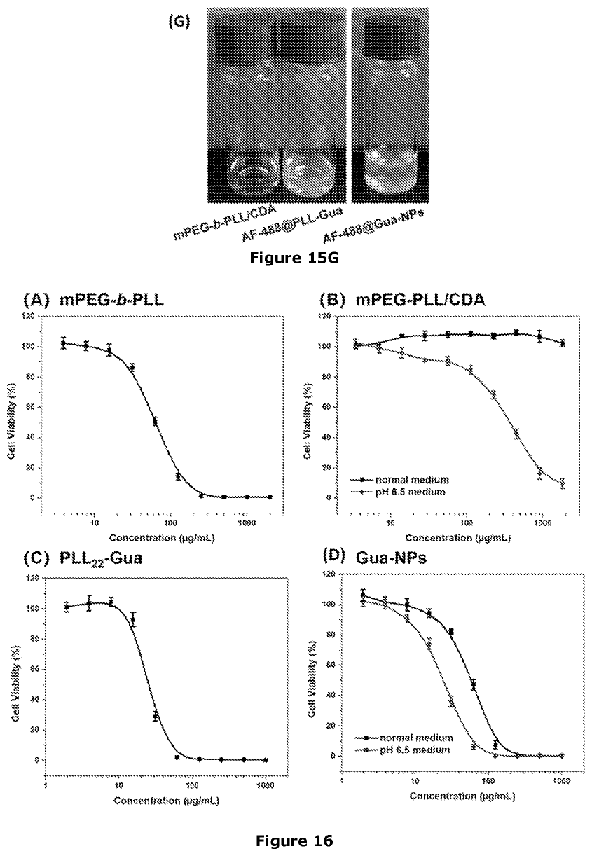

mPEG-b-PLG was unable to condense the positively charged polypeptides (PLL, PLL-Qua or PLL-Gua) into nanoparticles, resulting in precipitation after mixing with the cationic polypeptides (cloudy suspension, which was labelled as PLL-Mixture, Qua-Mixture or Gua-Mixture, respectively). The sizes of the mixtures were not uniform and multiple peaks were observed by dynamic light scattering measurements. In contrast, anionic mPEG-b-PLL/CDA and positively charged polypeptides (PLL, PLL-Qua, and PLL-Gua) self-assembled into uniform nanoparticles in aqueous solution, labelled as PLL-NPs, Qua-NPs, and Gua-NPs, respectively. The stability of these nanoparticles in PBS of pH 7.4 was studied. PLL-NPs were unstable as evidenced by the changes in the size distribution and precipitation in PBS of pH 7.4, limiting their application in vivo. mPEG-PLL/CDA and PLL₂₂-Gua self-assembled into nanoparticles (Gua-NPs) (FIG. 15D). As shown in FIG. 15A-C, Gua-NPs are stable in pH 7.4 PBS, and their sizes did not change over time, but they swelled or dissociated in a low pH environment (pH 6.6 and pH 5.4), indicating pH-sensitivity of the nanoparticles. In addition, the zeta potentials of Qua-NPs and Gua-NPs remained consistent at about 0 mV, throughout 24 h of incubation in PBS of pH 7.4 (FIGS. 15E and F), which is ideal for in vivo applications. These neutrally charged nanoparticles could facilitate intravenous administration in vivo. The increase in zeta potential of Qua-NPs and Gua-NPs in more acidic buffers (pH 6.6 and 5.4) indicated the dissociation of the nanoparticles and the release of cationic polypeptides (FIGS. 15E and F). The zeta potential changes of Gua-NPs in different pH buffer also indicated Gua-NPs were pH-sensitive. All these findings indicate the PLL₂₂-Gua can be released from the nanoparticles in the acidic tumor sites, but the nanoparticles remain stable when they circulate in the blood stream.

### In Vitro Cytotoxicity of Gua-NPs and PLL₂₂-Gua

The cytotoxicity of drug-free Gua-NPs was evaluated against BT474 cells. Although mPEG-PLL showed cytotoxicity (FIG. 16A), mPEG-PLL/CDA did not exhibit significant cytotoxicity at pH 7.4 even at the concentration of 1800 μg/mL and cell viability was above 95%. However, at pH 6.5, mPEG-PLL/CDA showed anticancer activity due to hydrolysis of CDA (FIG. 16B). The IC₅₀ (polymer concentration that leads to 50% inhibition of cell growth) of PLL₂₂-Gua was 25.1 (FIG. 16C), whereas the IC₅₀ of Gua-NPs at pH 6.5 and pH 7.4 was 24.2 and 60 μg/mL, respectively (FIG. 16D). These results further indicated that the nanoparticles responded to the low pH and thus release the anticancer polymer.

IC₅₀ values of p(lys-r-leu) and p(lys(Gua)-r-leu) were 52.1 and 26.4 μg/mL, respectively, against HepG2 human liver carcinoma cell line after incubation for 48 h (FIG. 21), demonstrating that the functionalization with guanidinium groups led to stronger anticancer activity.

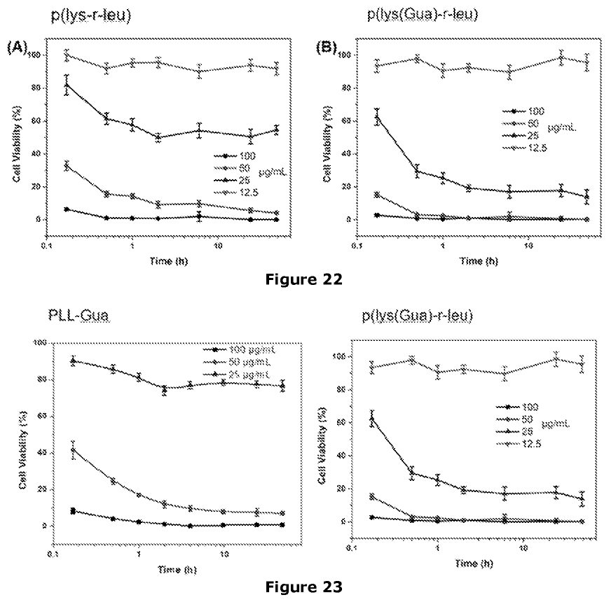

FIG. 22 shows the killing kinetic curves of p(lys-r-leu) and p(lys(Gua)-r-leu) against BT-474 human breast cancer cell line, demonstrating the functionalization with guanidinium groups also led to more rapid killing of cancer cells. Furthermore, the presence of the relatively hydrophobic leucine amino acid enhances killing kinetic of the polypeptide against the cancer cells (FIG. 23).

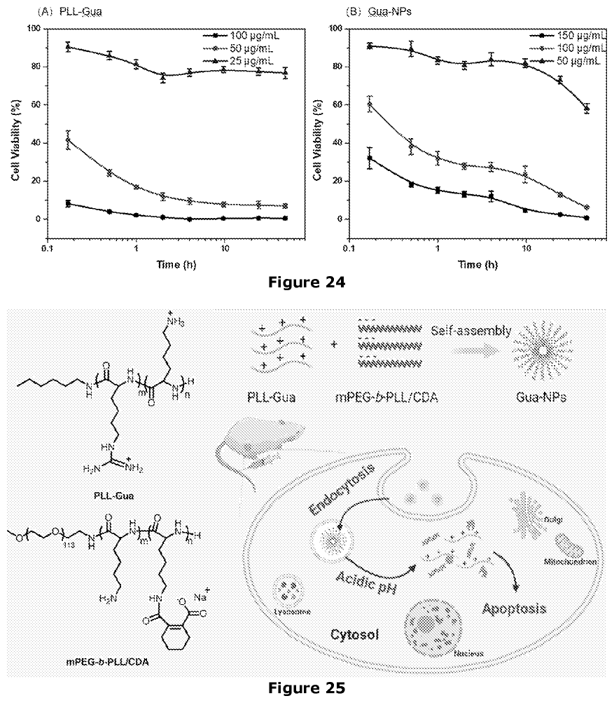

As shown in FIG. 24, PLL-Gua killed the cancer cells (BT-474) with a slower rate after being loaded into the nanoparticles. However, Gua-NPs still killed the cells effectively over 48 h (FIG. 16B), suggesting that Gua-NPs can exert a strong anticancer effect after being delivered to tumor tissues.

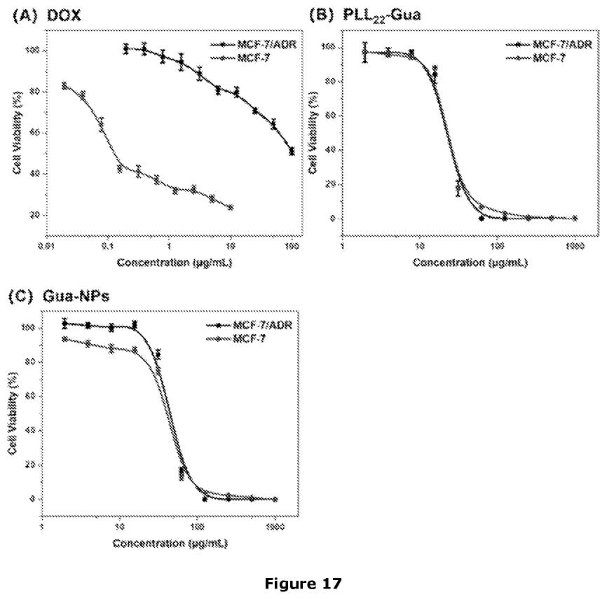

The multidrug resistance of the chemotherapeutic agents in clinic was an urgent problem which needs to be solved. MCF-7/ADR cells were highly resistant towards the small molecular chemotherapeutic drug DOX, and the IC₅₀ of DOX against MCF-7 and MCF-7/ADR was 0.13 and 100 μg/mL, respectively (FIG. 17A). The 769-fold difference of IC₅₀ value hinders the application of DOX in multidrug resistance tumors. However, the anticancer activity of PLL₂₂-Gua and Gua-NPs against MCF-7 and MCF-7/ADR was similar, indicating MCF-7/ADR cells were not resistant to the treatment with PLL₂₂-Gua or Gua-NPs (FIGS. 17B and C).

### In Vitro Anticancer Activity

Cytotoxicity of the anionic carrier mPEG-b-PLL/CDA was first evaluated at pH 7.4 using BT-474 cells. The cell viability remained above 95% even with polymer concentrations of up to 1800 μg/mL, indicating that the carrier was not cytotoxic under the simulated physiological conditions. In acidic tumor environments (e.g. pH 6.5), the polypeptide carrier showed some anticancer activity with IC50 of 368 μg/mL. This finding demonstrated that the anionic and pH-sensitive mPEG-b-PLL/CDA not only acted as a carrier to neutralize positively charged polymers, but also served as a chemotherapeutic agent. The cationic anticancer polypeptide PLL-Gua with guanidinium functional groups had stronger anticancer activity than PLL-Qua with quaternary ammonium groups, with IC50 value of 25.1 μg/mL (compared to 76.3 μg/mL) (FIGS. 26A and C). Both Gua-NPs and Qua-NPs showed anticancer activity at pH 7.4 (FIGS. 26B and D), implying that the anticancer polypeptides were able to be released from the nanoparticles. The anticancer activity of Gua-NPs was much stronger than that of Qua-NPs (IC50: 60.0 vs. 112.6 μg/mL at pH 7.4, FIGS. 26B and D). In addition, IC50 values of Qua-NPs and Gua-NPs at pH 7.4 were 2.7 and 2.5-fold higher than in the pH 6.5 medium, respectively. There are two possible reasons for the enhanced anticancer activity at the lower pH. Firstly, the anionic carrier responded to the lower pH environment, promoting the release of the anticancer polypeptides and thus increasing the anticancer effect of the cationic polypeptides. Secondly, the anionic polypeptide carrier exerted anticancer activity after releasing the anionic protecting groups at pH 6.5. As PLL-Gua and Gua-NPs demonstrated lower IC50 values and stronger anticancer efficacy, they were chosen for the following studies.

The killing kinetics of PLL-Gua and Gua-NPs against BT-474 at different concentrations were studied. As shown in FIGS. 26E and F, the cell viability after polypeptide treatments was dose- and time-dependent. An increased concentration led to faster killing kinetics. At the same concentrations (50 and 100 μg/mL), PLL-Gua killed the cancer cells more rapidly than its nanoparticle formulation due to the additional time required for the release of PLL-Gua from Gua-NPs.

### Mechanistic Study

To explore the anticancer mechanism of PLL-Gua and Gua-NPs, the cellular uptake of PLL-Gua labelled with AlexaFluor 488 (green) with and without nanoparticle formulation was first studied using confocal microscopy. Strong yellow-green fluorescence was seen in both AF-488@PLL-Gua solution and AF-488@Gua-NPs solution (FIG. 15G). In addition, the size distribution of AF-488@Gua-NPs in DI water was uniform (single peak by DLS measurements). As observed for Gua-NPs, the size of AF-488@Gua-NPs was also stable in PBS of pH 7.4 (FIG. 15A), but changed over time in low pH buffers (FIGS. 15B and C). Nucleus and endolysosomes were stained with Hoechst 33342 (blue) and LysoTracker Deep Red (red), respectively. As shown in FIG. 26H, Both PLL-Gua and Gua-NPs were readily taken up by BT-474 cells within 30 min. For PLL-Gua, only a small amount of green fluorescence was observed to overlap with red fluorescence after treatment, indicating little localization in the endolysosomes. For Gua-NPs, the green fluorescence was found to co-localize with red fluorescence of endolysosomes after 30 min of incubation, suggesting that Gua-NPs were taken up by the cells via endocytosis. This difference in cellular uptake mechanism could have also contributed to the observed differences in killing kinetics.

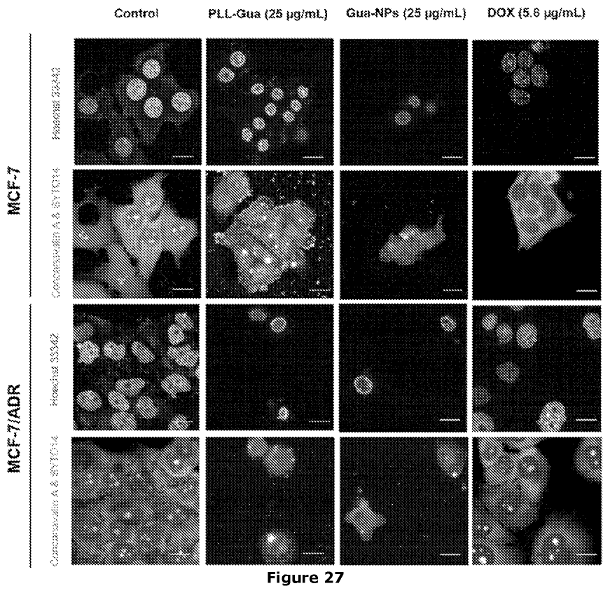

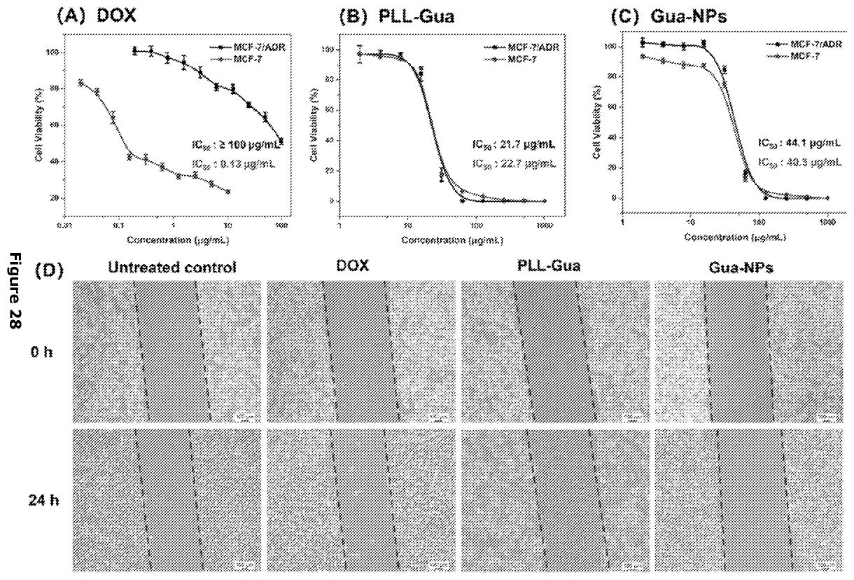

Next, the cellular morphology was studied before and after PLL-Gua or Gua-NPs treatment. Specifically, MCF-7 and MCF-7/ADR cells were treated with PLL-Gua (25 μg/mL) and Gua-NPs (25 μg/mL on PLL-Gua basis), and stained with 6 fluorescent dyes for the visualization of cellular components (FIG. 27). Untreated MCF-7 and MCF-7/ADR cells (control) exhibited normal epithelial morphology and polygonal shape. The 24 h treatment with PLL-Gua and Gua-NPs triggered changes to the morphology and cellular organization of MCF-7 cells, resulting in rounded cells with tightly packed organelles. Most notably, PLL-Gua and Gua-NPs caused a shrinkage in nuclei size and a decrease in staining intensity of the nucleoli. This suggests that PLL-Gua and Gua-NP induced nuclear condensation, which is characteristic of apoptotic cell death. DOX, a potent inducer of DNA damage, was used as a positive control. While DOX treatment (5.8 μg/mL) caused a drastic decrease in the staining of DNA and RNA in MCF-7 cells, it did not induce such changes in MCF-7/ADR cells. This shows that MCF-7/ADR cells were resistant to the effects of DOX. PLL-Gua and Gua-NPs induced similar morphological changes in MCF-7/ADR cells compared to MCF-7 cells, indicating their ability to bypass drug resistance mechanisms in the MCF-7/ADR cells.

Annexin V/PI staining apoptosis assay was then conducted to further explore the anticancer mechanism of PLL-Gua and Gua-NPs. Annexin V stains apoptotic cells and PI stains necrotic cells by penetrating the damaged cell membrane. Annexin VHighPILow, Annexin VHighPIHigh, and Annexin VLowPIHigh stand for early apoptosis, late apoptosis, and necrosis, respectively. As shown in FIGS. 26G and I, both PLL-Gua and Gua-NPs induced significant cell apoptosis rather than necrosis, and a higher population of early apoptotic cells were observed after the treatment at the lower polypeptide concentration (25 and 50 μg/mL for PLL-Gua and Gua-NPs, respectively) as compared to the control without any treatment (PLL-Gua vs control: 12.6±2.7% vs. 5.4±0.2%; Gua-NPs vs control: 11.2±2.6% vs. 5.4±0.2%). At the higher concentrations (50 and 100 μg/mL for PLL-Gua and Gua-NPs, respectively), a significantly higher population of late apoptotic cells were seen. These findings suggested that both PLL-Gua and Gua-NPs induced cancer cell death primarily based on an apoptotic mechanism. This is in contrast with the findings of quaternary ammonium-functionalized nanoparticles, where the nanoparticles induced necrosis in cancer cells. This might be due to membrane disruptive activity of quaternary ammonium-functionalized nanoparticles. These results implied that Gua-NPs were taken up by the cancer cells via endocytosis, and due to pH-sensitivity of Gua-NPs, PLL-Gua was subsequently released from the acidic endosome into the cytosol, where PLL-Gua performed its anticancer function, leading to cell apoptosis (FIG. 25).

### Drug Resistance and Migration Inhibition Study

The multidrug resistance of chemotherapeutic agents in clinic is a dire problem to be resolved. Here, drug-susceptible MCF-7 cells and drug-resistant MCF-7/ADR cells were used to evaluate PLL-Gua and Gua-NPs. MCF-7/ADR cells showed significant resistance towards the conventional chemotherapeutic drug DOX, and IC50 values of DOX against MCF-7 and MCF-7/ADR were 0.13 and 2 100 μg/mL, respectively (FIG. 28A). The 769-fold difference in IC50 value hinders the application of DOX for treatment of drug-resistant tumors. In sharp contrast, the anticancer efficacy of PLL-Gua and Gua-NPs against MCF-7 and MCF-7/ADR was almost equivalent, suggesting that drug-resistant MCF-7/ADR cells were susceptible to both PLL-Gua and Gua-NPs (FIGS. 28B and C).

Next, an in vitro wound scratch assay was performed to study the inhibition of MCF-7 cell migration by the anticancer agents. As shown in FIG. 28D, the untreated group showed a reduced wound gap after 24 h incubation, and a similar phenomenon was observed for MCF-7 cells treated with DOX. However, after incubation with PLL-Gua or Gua-NPs for 24 h, the wounds did not heal, suggesting that PLL-Gua and Gua-NPs prevented MCF-7 cell migration effectively.

### Hemolysis

Hemolytic activity has been widely used as an initial metric of toxicity for cationic polymers. Both PLL-Gua and Gua-NPs remained non-hemolytic even at high concentrations (HC10>4000 μg/mL), which is desirable for in vivo application.

### Evaluation of In Vivo Toxicity

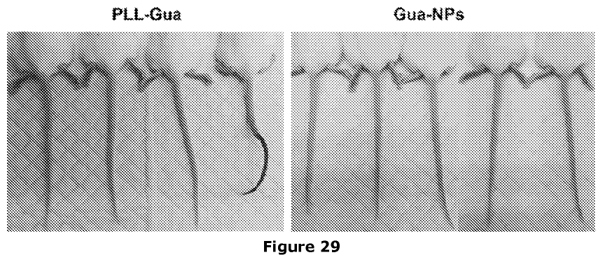

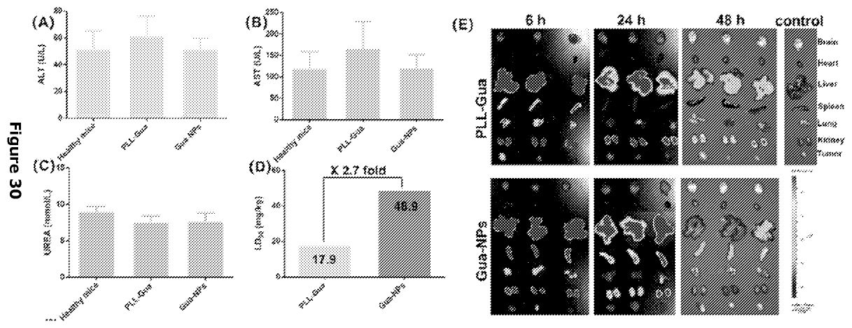

As safety is always the top priority, in vivo toxicity of PLL-Gua and Gua-NPs was further studied to evaluate clinical potential as anticancer agents. Healthy female Balb/c mice were randomly divided into 3 groups (n=5 in each group), followed by intravenous injection of PLLGua (10 mg/kg) or Gua-NPs (20 mg/kg, on PLL-Gua basis) on Day 0, 4, 8, 11, 15 and 18. The other five healthy mice served as the untreated control group. One of the mice in PLL-Gua group died, and PLL-Gua also gave rise to undesired blood coagulation in tail vein after repeated intravenous administrations (FIG. 29). The relatively higher toxicity would limit its application. Importantly, the mice treated with neutrally charged Gua-NPs tolerated the treatment well without any blockage of blood vessels, demonstrating Gua-NPs as a good delivery system for the cationic anticancer polypeptide.

All mice were sacrificed on Day 21. Blood was collected for complete blood count and blood chemistry assays. As shown in the results from the blood routine analysis (Table 3), there was no significant difference in the PLL-Gua and Gua-NPs treated mice compared with healthy ones, suggesting negligible acute inflammation and no signs of side effects caused by PLL-Gua or Gua-NPs. Additionally, the level of blood urea nitrogen (UREA), alanine aminotransferase (ALT) and aspartate aminotransferase (AST) were also within healthy ranges, suggesting no liver and kidney damage caused by PLL-Gua and Gua-NPs (FIG. 30A-C). It is also noteworthy that the Gua-NPs treatment did not cause obvious physical abnormalities, normal tissue damage, or body weight change, suggesting that Gua-NPs were well tolerated.

TABLE 3
Routine complete blood counts on Day 21 of healthy Balb/c mice
after intravenous injection with PLL-Gua (10 mg/kg) and Gua-NPs
(20 mg/kg on PLL-Gua basis) on Day 0, 4, 8, 11, 15 and 18.

| Entry | Control-Healthy mice | PLL-Gua | Gua-NPs |
|---|---|---|---|
| WBC (K/μL) | 9.43 ± 2.64 | 10.88 ± 1.64 | 8.42 ± 2.24 |
| NE (K/μL) | 2.86 ± 0.90 | 3.08 ± 0.37 | 2.43 ± 0.61 |
| LY (K/μL) | 4.95 ± 1.20 | 6.26 ± 1.05 | 4.75 ± 1.32 |
| MO (K/μL) | 0.75 ± 0.20 | 0.72 ± 0.09 | 0.62 ± 0.18 |
| EO (K/μL) | 0.62 ± 0.31 | 0.62 ± 0.18 | 0.45 ± 0.16 |
| BA (K/μL) | 0.25 ± 0.11 | 0.20 ± 0.08 | 0.17 ± 0.06 |
| RBC (M/dL) | 10.06 ± 0.32 | 10.47 ± 0.14 | 10.37 ± 0.33 |
| HCT (%) | 63.70 ± 2.24 | 65.54 ± 0.91 | 64.48 ± 2.44 |
| MCV (fL) | 63.34 ± 0.74 | 62.62 ± 1.20 | 62.22 ± 0.93 |
| MCH (pg) | 12.2 ± 0.27 | 11.42 ± 0.16 | 11.40 ± 0.19 |
| MCHC (g/dL) | 19.24 ± 0.29 | 18.26 ± 0.36 | 18.32 ± 0.38 |
| RDW (%) | 17.64 ± 0.60 | 18.46 ± 0.50 | 18.16 ± 0.35 |
| PLT (K/μL) | 278 ± 67 | 329 ± 57 | 270 ± 99 |
| MPV (fL) | 5.54 ± 0.23 | 5.58 ± 0.26 | 5.66 ± 0.26 |

WBC: White Blood Cells; NE: Neutrophils; LY: Lymphocytes; MO: Monocytes; EO: Eosinophils; BA: Basophil; RBC: Red Blood Cell; HCT: Hematocrit; MCV: Mean Corpuscular Volume; MCH: Mean Corpuscular Hemoglobin; MCHC: Mean Corpuscular Hemoglobin Concentration; RDW: Red blood cell Distribution Width; PLT: Platelets; MPV: Mean Platelet Volume.

All the results from toxicity studies indicated that Gua-NPs are a potential candidate for cancer treatment in vivo.

### LD50 Determination of PLL-Gua and Gua-NPs

Acute toxicity of PLL-Gua and Gua-NPs was evaluated by determining their LD50 value, which is the dose that causes death in 50% of the treated mice during a given period. PLL-Gua and Gua-NPs were injected intravenously via tail veil, and LD50 values were determined to be 17.9 and 48.9 mg/kg (on PLL-Gua basis), respectively (FIG. 30D). The nanoparticle formulation reduced PLL-Gua's toxicity, and its LD50 was increased by 2.7 times by neutralizing the cationic charges in PLL-Gua. These findings suggest that the nanoparticles are a promising formulation for in vivo delivery of the anticancer agent PLL-Gua.

### In Vivo Biodistribution

Fluorescence imaging has been widely used to study biodistribution/EPR effect of drug-loaded nanoparticles. To study the in vivo distribution of PLL-Gua and Gua-NPs, Balb/c nude mice bearing subcutaneous BT-474 tumors were subjected to ex vivo fluorescence imaging study. Specifically, NIR dye Alexa Fluor 750-labelled PLL-Gua and Alexa Fluor 750-labelled Gua-NPs were injected intravenously via tail veil. At different time intervals after administration, the mice were euthanized, and tumors and major organs (brain, heart, liver, spleen, lungs, and kidneys) were excised and imaged. As shown in FIG. 30E, the fluorescence intensity at the tumors treated with PLL-Gua and Gua-NPs was similar and had no significant difference at 6 h after injection. However, at 24 h post injection, the mice treated with Gua-NPs showed significantly higher fluorescence at the tumor site compared to those treated with free PLL-Gua as no fluorescence was seen in the tumors treated with PLLGua. These results demonstrated that Gua-NPs accumulated in the tumor site more effectively than PLL-Gua, owing to the EPR effect of Gua-NPs. In addition, the fluorescence signals of Alexa Fluor 750-labelled PLL-Gua and Gua-NPs weakened over time due to clearance from the organs. Both PLL-Gua and Gua-NPs treated groups showed no tumor accumulation at 48 h after injection. The remaining Gua-NPs were mostly sequestered into the spleen, whereas accumulation in brain, heart and lungs was negligible. Free polypeptide PLL-Gua was mostly excreted by the liver and kidneys. Some fluorescence signal was seen in the brain tissues of the mice treated with the free polypeptide PLL-Gua at 6 h post treatment, while it was not observed in the brain tissues of mice treated with Gua-NPs, showing that the nanoparticle delivery system is advantageous in minimizing accumulation in brain tissues. In addition, most Gua-NPs were cleared from the body after 48 h post administration, which is likely the reason for the non-toxicity observed even the cumulative doses of Gua-NPs over the treatment period of 21 days (6×20 mg/kg of mouse body weight=120 mg/kg of mouse body weight) was higher than LD50 (48.9 mg/kg of mouse body weight) (FIG. 30A-C).

### In Vivo Antitumor Efficacy of Gua-NPs

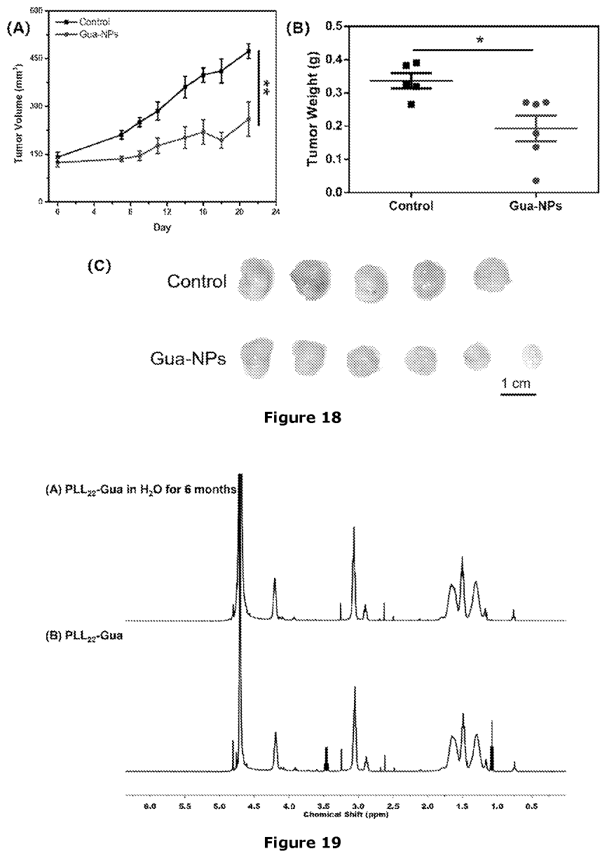

As shown in FIG. 18A, Gua-NPs significantly suppressed the tumor growth as compared to the untreated control group, and the tumor suppression rate of Gua-NPs treated group on Day 21 was 45.1%. Tumor weight measurements (FIG. 18B) and the photos of dissected tumors (FIG. 18C) at the end of the treatment also showed a therapeutic benefit with the Gua-NPs treatment. These findings indicate that Gua-NPs are a potential candidate for cancer treatment.

The lower in vivo toxicity and more effective tumor accumulation of Gua-NPs supported further investigations of the therapeutic efficacy of the nanoparticles in vivo. Tumor-bearing Balb/c nude mice (tumor volume: −130 mm³) were randomly distributed into 2 groups on Day 0: Control and Gua-NPs (20 mg/kg, on PLL-Gua basis). Gua-NPs were intravenously injected on Day 0, 2, 7, 9, 14 and 17. Drug-free Gua-NPs significantly suppressed tumor growth over 21 days in comparison with the control (FIG. 18A). On Day 21, tumors were excised and weighed. Reduced tumor weight in the treated mice showed the therapeutic benefit of Gua-NPs (FIGS. 18B and C).

To further validate the therapeutic effect, the excised tumors were processed for H&E and TUNEL staining for cell morphology observation and apoptosis analysis. The Gua-NPs treated mice induced significantly greater necrosis and apoptosis relative to the untreated control. All these results indicated that Gua-NPs are a potentially promising candidate for cancer treatment.

### Degradation

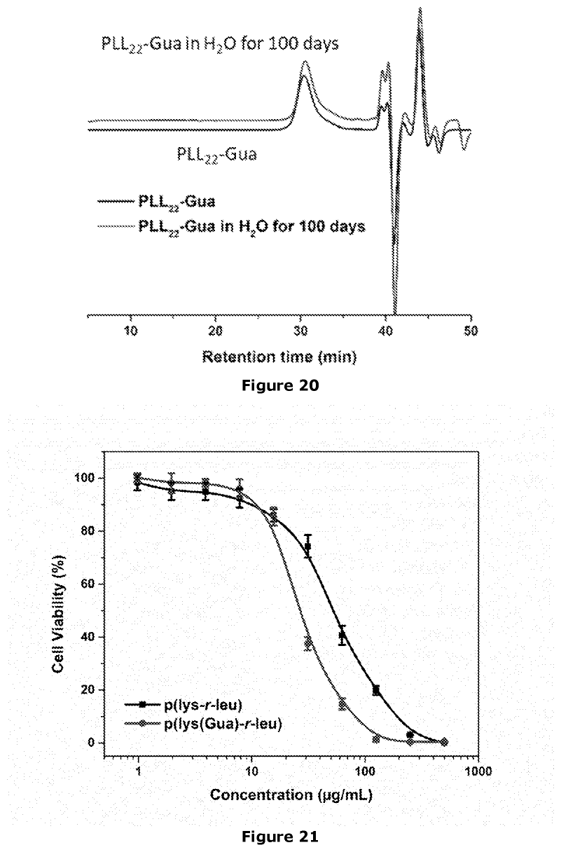

After PLL₂₂-Gua was kept in water for 6 months at room temperature, and its ¹H NMR spectrum did not change (FIG. 19). Also, the GPC trace of PLL₂₂-Gua after incubation at 37° C. for 100 days was also similar to the original one (FIG. 20). These findings demonstrate that the guanidinium-functionalized polypeptide is stable and can be stored for a long time.

### CONCLUSION

Guanidinium-functionalized polypeptides were synthesized. Particularly, PLL-Gua showed excellent broad-spectrum antimicrobial efficacy against S. aureus, MRSA, E. coli, A. baumannii, P. aeruginosa, K. pneumoniae and C. albicans, enhanced killing efficiency and fast killing kinetics as compared to the other polypeptides reported in this study. In addition, PLL-Gua did not exhibit hemolytic activity even at the concentration of 4000 μg/mL. More importantly, PLL₂₂-Gua was proven to be a potent antimicrobial agent in the treatment of MRSA-induced wound infection in a murine model, without inducing acute dermal toxicity. The polypeptide PLL₂₂-Gua is a promising macromolecular antimicrobial agent for the prevention and treatment of MRSA-caused skin infection. The excellent anticancer efficacy of PLL₂₂-Gua and Gua-NPs in vitro and in vivo demonstrates that the PLL₂₂-Gua is also a potential candidate for cancer treatment. Synthetic anticancer polypeptide PLL-Gua successfully self assembled with the negatively charged polypeptide carrier mPEG-b-PLL/CDA to form the neutrally charged nanoparticles Gua-NPs. Gua-NPs showed excellent stability in PBS (pH 7.4) and pH-sensitivity in an acidic environment (pH 6.5). Gua-NPs exhibited similar in vitro anticancer efficacy at pH 6.5 as PLL-Gua. Both PLL-Gua and Gua-NPs induced cell apoptosis in a dose-dependent manner. PLL-Gua entered cells likely through membrane translocation, while Gua-NPs were taken up by the cells via endocytosis. Although MCF-7/ADR cells were resistant to DOX, they were susceptible to PLL-Gua and Gua-NPs. PLL-Gua and Gua-NPs triggered similar changes to the morphology and cellular organization of MCF-7 and MCF-7/ADR cells, demonstrating their ability to bypass drug resistance mechanisms in MCF-7/ADR cells. In addition, Gua-NPs presented lower in vivo toxicity with higher LD50 value, and increased accumulation at the tumor site as compared to free PLL-Gua without nanoparticle formulation. Furthermore, Gua-NPs prevented cancer cell migration in vitro. More importantly, the drug-free neutrally charged Gua-NPs demonstrated excellent antitumor efficacy in a BT-474 human breast cancer xenograft mouse model with negligible toxicity at the doses tested. The delivery of synthetic cationic anticancer polypeptide using a pH-sensitive anionic polypeptide is a promising approach for the treatment of cancer to overcome drug resistance.

It will be appreciated that many further modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Throughout this specification and the claims which follow, unless the context requires otherwise, the phrase “consisting essentially of”, and variations such as “consists essentially of” will be understood to indicate that the recited element(s) is/are essential i.e. necessary elements of the invention. The phrase allows for the presence of other non-recited elements which do not materially affect the characteristics of the invention but excludes additional unspecified elements which would affect the basic and novel characteristics of the method defined.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A functionalised polypeptide of Formula (I) or a pharmaceutically acceptable salt, solvate, stereoisomer or prodrug thereof, comprising:
a) monomeric units of Formula (A) 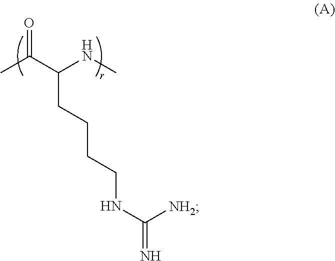
b) at least one monomeric unit of Formula (B) 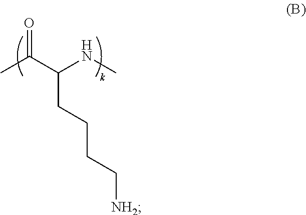
wherein
m is an integer from 5 to 100; and
k is an integer from 1 to 20;
wherein a percentage of monomeric units of Formula (A) relative to the functionalised polypeptide of Formula (I) is about 50% to about 98%.

2. The functionalised polypeptide according to claim 1, wherein the monomeric units of Formula (A) and at least one monomeric unit of Formula (B) form a random co-polypeptide; and
wherein the functionalised polypeptide of Formula (I) is characterised by a percentage of monomeric units of Formula (A) relative to the functionalised polypeptide of Formula (I) of about 95%.

3. (canceled)

4. The functionalised polypeptide according to claim 1, wherein the functionalised polypeptide of Formula (I) further comprises at least one monomeric unit of Formula (C): 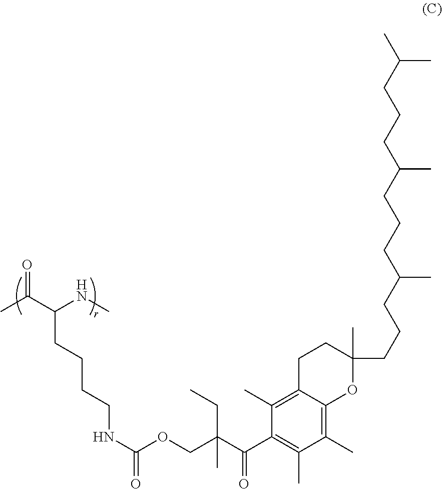
wherein g is an integer from 1 to 10.

5. (canceled)

6. The functionalised polypeptide according to claim 4, wherein the functionalised polypeptide of Formula (I) is characterised by a percentage of monomeric units of Formula (C) relative to the functionalised polypeptide of Formula (I) of about 5% to about 20%; and
wherein the monomeric units of Formula (C), monomeric units of Formula (A) and at least one monomeric unit of Formula (B) form a random co-polypeptide.

7. (canceled)

8. The functionalised polypeptide according to claim 4, wherein m is 18, k is 2, and g is 2, or m is 30, k is 3, and g is 2.

9. The functionalised polypeptide according to claim 4, wherein when monomeric units of Formula (C) are present, the functionalised polypeptide of Formula (I) is characterised by a micelle diameter of about 10 nm to about 200 nm, and/or a critical micelle concentration (CMC) value of about 1 μg/mL to about 100 μg/mL, and/or by a zeta potential (ζ) of about +1 mV to about +30 mV.

10. (canceled)

11. The functionalised polypeptide according to claim 1, wherein the functionalised polypeptide of Formula (I) further comprises monomeric units of Formula (D): 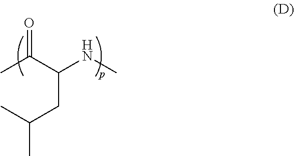
wherein p is an integer from 5 to 100.

12-13. (canceled)

14. The functionalised polypeptide according to claim 11, wherein m is 27, k is 2, and p is 12.

15. The functionalised polypeptide according to claim 11, wherein the functionalised polypeptide of Formula (I) is characterised by at least one of the following:
a) a degree of polymerisation of 5 to 100; and
b) a polydispersity index (PDI) of about 1.1 to about 1.7.

16. (canceled)

17. The functionalised polypeptide according to claim 1, wherein the functionalised polypeptide of Formula (I) is characterised by an antimicrobial activity against Gram-positive bacteria, Gram-negative bacteria and fungi;
wherein the Gram-positive bacteria is selected from S. aureus and MRSA, the Gram-negative bacteria is selected from E. coli, A. baumannii, P. aeruginosa and K. pneumonia, and the fungi is C. albican.

18. (canceled)

19. The functionalised polypeptide according to claim 1, wherein the functionalised polypeptide of Formula (I) is characterised by at least one of the following:
a) an antimicrobial activity with a minimum inhibition concentration (MIC) of about 1 μg/mL to about 200 μg/mL;
b) a minimum bactericidal concentration or minimum fungicidal concentration (MBC or MFC)/MIC ratio (R) of about 1 to about 500:
c) a minimum haemolytic concentration at 50% lysis (HC₅₀) of about 100 μg/mL to about 8000 μg/mL, and/or by a haemolytic concentration (HC₅₀) of about 4000 μg/mL to about 8000 μg/mL:
d) an IC₅₀ value of about 1 μg/mL to about 200 μg/mL against cancer cells; and
e) an IC₅₀ ratio of human breast cancer cell line (MCF-7) relative to drug-resistant human breast cancer cell line (MCF-7/ADR) of about 1.

20-23. (canceled)

24. A functionalised polypeptide nanoparticle, comprising:
a) functionalised polypeptides of Formula (I) or a pharmaceutically acceptable salt, solvate, stereoisomer or prodrug thereof according to claim 1; and
b) functionalised polypeptides of Formula (II) or a pharmaceutically acceptable salt, solvate, stereoisomer or prodrug thereof, comprising
i) monomeric units of Formula (E): 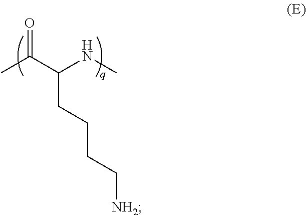
wherein q is an integer from 0 to 10;
ii) monomeric units of Formula (F): 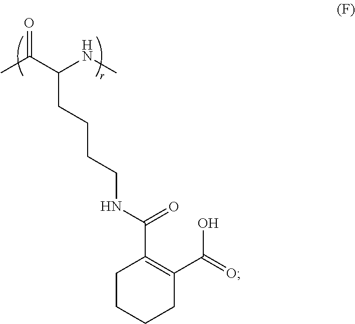
wherein r is an integer from 10 to 100; and
iii) a methoxy poly(ethylene glycol) (mPEG) terminal end;
wherein a percentage of monomeric units of Formula (F) relative to the functionalised polypeptide of Formula (II) is about 50% to about 98%;
wherein a mole ratio of functionalised polypeptides of Formula (I) to functionalised polypeptides of Formula (II) is about 1:1 to about 1:10.

25. The functionalised polypeptide nanoparticle according to claim 24, wherein q is an integer from 1 to 8, r is an integer from 15 to 50, and q is 6 and r is 20.

26. The functionalised polypeptide nanoparticle according to claim 24, wherein the monomeric units of Formula (E) and monomeric units of Formula (F) form a random co-polypeptide; and/or
wherein the functionalised polypeptide nanoparticle is dissociable at a pH of about 1 to less than 7.

27. The functionalised polypeptide nanoparticle according to claim 24, wherein the mPEG terminal end has a molecular weight of about 2000 g/mol to about 10,000 g/mol; and/or is characterised by percentage of monomeric units of Formula (F) relative to the functionalised polypeptide of Formula (II) of about 80 to about 90%.

28-29. (canceled)

30. The functionalised polypeptide nanoparticle according to claim 24, wherein the functionalised polypeptide nanoparticle is characterised by at least one of the following:
a) an IC₅₀ value of about 1 μg/mL to about 200 μg/mL against cancer cells;
b) an IC₅₀ value at a pH of about 6.5 of about 10 μg/mL to about 50 μg/mL against cancer cells:
c) an IC₅₀ value at a pH of about 7.4 of about 25 μg/mL to about 150 μg/mL against cancer cells; and
d) an IC₅₀ ratio of human breast cancer cell line (MCF-7) relative to drug-resistant human breast cancer cell line (MCF-7/ADR) of about 1.

31-32. (canceled)

33. A pharmaceutical composition comprising an effective amount of a functionalised polypeptide of formula (I) according to claim 1 or a functionalised polypeptide nanoparticle according to claim 24 or a pharmaceutically acceptable salt, solvate, stereoisomer or prodrug thereof, wherein the pharmaceutical composition optionally further comprises a pharmaceutically acceptable carrier, excipient or diluent.

34. (canceled)

35. A method for treating a microbial infection and/or cancer, the method comprising administering to a subject in need thereof a therapeutically effective amount of a functionalised polypeptide of formula (I) according to claim 1 or a functionalised polypeptide nanoparticle according to claim 24 or a pharmaceutically acceptable salt, solvate, stereoisomer or prodrug thereof.

36. The method according to claim 35, wherein the microbial infection is a bacterial infection or a fungal infection.

37-39. (canceled)

40. The method according to claim 35, wherein the cancer is a drug resistant cancer, breast cancer, or a tumorous cancer.

41-44. (canceled)
